(19) United States
(12) Patent Application Publication
(10) Pub. No.: US 2017/0298444 A1
(43) Pub. Date: Oct. 19, 2017

(54) METHODS FOR PREDICTING THE RISK OF DEVELOPING BREAST CANCER

(71) Applicant: Dana-Farber Cancer Institute, Inc., Boston, MA (US)

(72) Inventors: Shailja PATHANIA, Boston, MA (US); David LIVINGSTON, Brookline, MA (US)

(21) Appl. No.: 15/514,152

(22) PCT Filed: Sep. 24, 2015
(86) PCT No.: PCT/US2015/051926

§ 371 Date: Mar. 24, 2017

Related U.S. Application Data
(60) Provisional application No. 62/054,787, filed on Sep. 24, 2014.

(51) Int. Cl. C12Q 1/68; G01N 33/574; G06F 19/00; G01N 21/64

(52) U.S. Cl. CPC C12Q 1/6886; G06F 19/3431; G01N 21/6428; G01N 33/57496; C12Q 2600/156; C12Q 2600/118; G01N 2333/9108; G06F 19/345; G01N 2021/6439

(57) ABSTRACT

The present invention provides methods for accessing the risk of developing breast cancer.

### RELATED APPLICATIONS

This application claims priority to, and the benefit of, U.S. Provisional Application No. 62/054,787 filed on Sep. 24, 2014, the contents of which are incorporated here by reference in its entirety.

### GOVERNMENT INTEREST

This invention was made with government support under P01 CA080111. awarded by the National Institutes of Health. The government has certain rights in the invention.

### FIELD OF THE INVENTION

The present invention relates generally to an assay for accessing a subject's risk for developing of breast cancer.

### BACKGROUND OF THE INVENTION

Currently, tools to predict BRCA1 mutation driven breast cancer are based on genetic information about the mutation, personal and family history of the patient and/or on the BRCA1. functional assays. So far, all the functional assays are based on introducing the mutant BRCA1 protein in cell lines and then testing the function of this mutant BRCA1 in these cells. These functional assays are not only time consuming and labor intensive but given that they are performed in cell lines and/or yeast or mouse system, they also do not truly reflect what is going on in the cells of the woman bearing that mutation. Furthermore, in these functional assays, rare variants are rarely tested. Accordingly a need exists for a predictive assay that is rapid and reliable. The present invention fulfills that need.

### SUMMARY OF THE INVENTION

In various aspects the invention provides methods for assessing the risk of developing cancer in a subject having a germline mutation in a gene known to be associated with cancer by providing a mitotic non-tumor cell from the subject; culturing the cell to obtain a subject cell population; labeling the subject cell population with a first detectable label; co-culturing the subject cell population with a control cell population labeled with a second detectable label; exposing the cell populations to a DNA damaging agent; and determining the ratio of cells having the first detectable label to cells having the second detectable label. When the ratio is less than 1 the subject has an increased risk of developing cancer.

The germline mutation is a BRAC1 or BRAC2 mutation. The non-tumor cell is a fibroblast such as a skin cell. The control population is derived from a subject not having the germline mutation. The subject cell population and the control cell population are the same type of cells.

The DNA damaging agent is for example, cisplatin, hydroxyurea ultraviolet radiation or 4-nitro-quinoline. The detectable label is a fluorescent dye.

The ratio of the first detectable label and the second detectable label is by FACS. In some aspects the invention further includes making a clinical management recommendation for the patient. The clinical management recommendation for example is that the subject receives prophylactic therapy for said cancer.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1. Primary fibroblast and HMEC strains (BRCA1^{+/+} and BRCA1^{mut/+}) used in this study. (a) 22 primary fibroblast strains were derived from skin punch biopsies and 15 primary, mammary epithelial cell (HMECs) strains from prophylactic mastectomies performed on BRCA1 mutation carrying (BRCA1^{mut/+}) women. BRCA1^{+/+} control HMECs (n=7) were derived from reduction mammoplasty tissue, and control fibroblasts (n=10) were derived from skin punch biopsies from women lacking BRCA1 mutations. (b) Distribution of BRCA1 mutations in primary fibroblasts and HMECs. (c) Western blot analysis of total BRCA1 protein levels in BRCA1^{mut/+} and BRCA1^{+/+}HMEC lines. Equivalent amounts of whole-cell lysate (prepared in NETN300) were loaded and probed with anti-BRCA1 monoclonal Ab (SD118). GAPDH served as a loading control. (d) Western blot analysis of BRCA1 protein levels in the nuclear fraction of BRCA1^{mut/+} and BRCA1^{+/+} fibroblast lines. Cells were pre-lysed in pre-extraction buffer, and the pellet was re-suspended in NETN400 buffer to prepare the nuclear extract. The intense BRCA1 band in 83 (185delAG) is likely the previously discovered truncated product of this mutant allele⁵¹.

FIG. 2. Spindle pole formation, centrosome number, checkpoint activation, and Rad51 recruitment to DSB (a) Representative images of HMECs (left panel) and skin fibroblasts (right panel), from BRCA1 mutation carriers (BRCA1^{mut/+}) and wild type BRCA1 counterparts (BRCA1^{+/+}) were immunostained with ant-TPX2 Ab to detect spindles. N=50 spindles were analyzed for each line. (b) Centrosome number was determined by immunostaining HMECs (left panel) and fibroblasts (right panel) with Ab to γ-tubulin. N=50 cells for each line were counted and cells with centrosomes ≦2 were considered normal. (c) S-phase checkpoint in response to UV and IR-induced DNA damage in control and BRCA1^{mut/+} strains. Three BRCA1^{+/+} (AR7, CP22 and CP29) and three BRCA1^{mut/+} HMEC strains (79, CP10 and CP16) were irradiated with increasing doses of UV (left panel). Two hours later, they were pulse-labeled with BrdU for 30 minutes. The cells were then harvested, stained with BrdU antibody, and processed for FACS. For IR-induced S-phase checkpoint analysis (right panel), cells were irradiated with IR (10Gy), pulse labeled 2 hours post damage for 30 minutes with BrdU. Cells were then harvested and processed for FACS (red histograms). Un-irradiated cells (0Gy, blue histograms) were used as controls. Error bars indicate standard deviation between the results of three, independent experiments. (d) G2/M checkpoint activation in response to UV and IR-induced DNA damage in BRCA1^{mut/+} and control cells. BRCA1^{+/+} and BRCA1^{mut/+} cells were irradiated with either UV (10 J/m2) or IR (10Gy), allowed to recover for 2 hours and then harvested for FACS analysis. Percentage of cells in mitosis was determined by staining cells with propidium iodine (PI) and Alexa 488-conjugated phosphorylated histone H3 (S28) antibody. Mock-irradiated (-dam) cells served as controls. (e) HMECs and (f) fibroblasts, derived from BRCA1 mutation carriers (BRCA1^{mut/+}) and BRCA1^{+/+} individuals were exposed to IR (10Gy) and allowed to recover for 4 hrs. Cells were fixed cells and coimmunostained with Abs to γ-H2AX and Rad51. Graphs depicting the fraction of cells with Rad51 foci co-localized with γ-H2AX for each line are plotted for both HMECs and fibroblasts (right panels in e and f). Means and standard deviations of at least three experiments for each strain are shown. Green columns=wt BRCA1^{+/+} and red columns=BRCA1^{mut/+} lines.

FIG. 3. FACS based cell survival assay shows that HR-DSBR is not defective in BRCA1^{mut/+} cells. (a) FACS-based cell survival assay was used to determine the sensitivity of cells to various DNA damage inducing agents. BRCA1^{mut/+} and BRCA1^{+/+} strains were ‘color-coded’ by immortalizing with an htert (ht) retro vector that lacked or contained a GFP reporter. These cells were co-plated and exposed to DNA damaging agents. Cells were allowed to recover for 8 days before harvesting for FACS analysis. Cell survival data is plotted as a ratio of GFP positive to GFP negative cells. Ratio between Wt/Wt (Green), Mutant/Mutant (Blue) and Mutant/WT (Red) is plotted in the graphs below. Error bars were calculated as the standard error propagation (SEP) in the ratios of each of the combinations in three independent experiments. (b) Combinations of BRCA1^{mut/+} and BRCA1^{+/+} HMECs were exposed to different concentrations of PARP inhibitor, and the ratio of each of these combinations was plotted (left). The average ratio of WT/WT, Mut/Mut and Mut/WT was also calculated and plotted (right). (c) (Left) Combinations of BRCA1^{mut/+} and BRCA1^{+/+} fibroblasts were exposed to different concentrations of PARP inhibitor, and the survival ratio of each of these combinations was plotted (left). An average ratio of WT/WT, Mut/Mut and Mut/WT was also calculated and plotted (right). (d) U20S cells (containing or lacking a GFP reporter) were infected with lentiviral vectors encoding an shRNA directed at Luciferase (ShLuc, control) or at BRCA1 (shBRCA1). Green=ShLuc/ShLuc, Blue=shBRCA1/shBRCA1, and Red=shBRCA1/ShLuc. Averages of the results of individual experiments are plotted. (e) BRCA1^{mut/+} (CP10 and CP16) were transduced with shRNA directed at GAPDH (siGAPDH), or BRCA1 (siBRCA1). 3 days post transfection, combinations of siGAPDH or siBRCA1-transduced BRCA1^{mut/+} HMECs (CP10 and CP16) were co-plated with AR7 (a BRCA1^{+/+} HMEC) and exposed to various doses of PARP inhibitor. Averages of the results generated by these combinations were plotted.

FIG. 4. BRCA1^{mut/+} cells are defective in generation of phospho-RPA32-coated ssDNA. (a) phospho-RPA32 (pRPA32) loading on chromatin is BRCA1 dependent. U20S cells infected with lentiviral shRNA directed at BRCA1 (ShB) exhibit reduced pRPA32 loading after HU-induced stalled fork formation. (b) After UV induced DNA damage BRCA1^{mut/+} fibroblasts exhibit reduced pRPA32 loading on ssDNA, compared to BRCA1^{+/+} lines. Cells were irradiated with 30 J/m2 of UV and harvested 3 hours post damage. Chromatin extracts were prepared, and the relevant western blot was probed with antibody to phosphorylated RPA32. The replication status for each line was checked on the day of the experiment by BrdU uptake measurement, and only those lines which showed similar replication profiles were analyzed in single gel. A subset of lines tested is shown here. Western blots for other WT and BRCA1 mutant lines are shown in FIG. 10. (c) BRCA1^{mut/+} fibroblasts reveal reduced pRPA32 loading on ssDNA compared to BRCA1^{+/+} lines, after HU exposure (10 mM for 3 hrs). Protein-containing extracts were prepared as described above. (d) BRCA1^{mut/+}HMECs reveal reduced pRPA32 loading on ssDNA, compared to BRCA1^{+/+} HMECs after UV irradiation. (e) BRCA1^{mut/+} cells efficiently recruit RPA32 to DSBs. RPA32 loading at laser-induced DSBs was equivalently efficient in BRCA1^{mut/+} and BRCA1^{+/+} lines. Laser micro-irradiation was performed, and 1 hr later, cells were fixed. Cells were co-stained with anti-γ-H2AX to reflect the existence DSBs. (f) BRCA1^{mut/+} skin fibroblasts (068), and (g) mammary epithelial cells (CP17), each infected with a lentiviral vector expressing HA-tagged BRCA1, were either irradiated with 10Gy IR (upper panel) or 30 J/m2 of UV (lower panel) through a micropore membrane, and allowed to recover for 3 hrs. Cells were co-immunostained with Abs to BRCA1 and HA. (h, i) Phospho-RPA32 recruitment to ssDNA was analyzed with a subset of primary BRCA1^{mut/+} and BRCA1^{+/+} firboblasts (h) and HMECs (i), infected with a lentiviral vector expressing either full length WT BRCA1 (HA-tagged) or eGFP (control). Cells were irradiated with 30 J/m2 UV, harvested 3 hrs later, and used to prepare chromatin-rich extracts. Western blots were immunostained with Ab to phospho-RPA32.

FIG. 5. Heterozygous BRCA1^{mut/+} cells reveal increased numbers of DNA breaks after stalled fork-inducing DNA damage, and are more sensitive than WT BRCA1^{+/+} cells to stalled fork-inducing agents. (a) BRCA1^{mut/+} HMECs are prone to increased fork collapse compared to BRCA1^{+/+} cells, after exposure to a stalled fork-inducing agent (UV). (b) Skin fibroblasts, derived from BRCA1 mutation carriers (BRCA1^{mut/+}) and wild type BRCA1 counterparts (BRCA1^{+/+}), were irradiated with low dose UV (5 J/m2) and allowed to recover for 18 hrs. Cells were immunostained with Ab to 53BP1 (a marker for collapsed replication forks). The R panel depicting the percentage of cells with ≧10 53BP1 foci per cell is plotted for HMECs and fibroblasts. Means and standard deviations of at least three experiments for each strain are shown. Green bars=wt BRCA1^{+/+} and red bars=BRCA1^{mut/+} strains. All strains within each cell type revealed similar BrdU uptake profiles (data not shown). (c) Heterozygous BRCA1^{mut/+} cells reveal a compromised DNA repair efficiency compared to WT BRCA1^{+/+} cells after exposure to low dose UV (5 J/m2). DNA damage was measured as a percentage of DNA in comet tails after UV-induced DNA damage. Representative images for comets in unirradiated (−UV) and irradiated (+UV) samples are shown. (d) BRCA1^{mut/+} and BRCA1^{+/+} HMECs were irradiated with 5 J/m2 of UV and allowed to recover for 3 hours before carrying out the alkaline comet assay. Percentage of DNA in the comet tails is plotted for unirradiated (left panel) and irradiated cells (right panel). Green bars=BRCA1^{+/+} and red=BRCA1^{mut/+} cells. The mean result and standard deviation of at least three different experiments is plotted. In each experiment at least 250 individual cells were scored for percentage of DNA in the comet tails using CellProfiler software. (e, f) (Left panels) Combinations of BRCA1^{mut/+} and BRCA1^{+/+} HMECs (e) and fibroblasts (f) were irradiated with different doses of UV. (Right) Average of data plotted on left. (g, h) Combinations of BRCA1^{mut/+} and BRCA1^{+/+} HMECs (g) and fibroblasts (h) were incubated with increasing concentrations of cisplatin for 15 hours. Cells were allowed to recover for 6 days and then harvested for FACS analysis. Panels on the right show the averages of data plotted on the left.

FIG. 6. Evidence for conditional haploinsufficiency for DSBR in BRCA1^{mut/+} HMECs after pre-exposure to a stalled fork inducing agent.

(a) Recruitment of Rad51 to IR-induced DSBs is reduced in heterozygous BRCA1^{mut/+}, and not in WT BRCA1^{+/+} HMECs when pre-exposed to stalled fork-inducing damage. HMECs derived from a BRCA1 mutation carrier (CP16, BRCA1^{mut/+}) and a wt counterpart (CP29, BRCA1^{+/+}) were irradiated with different doses of UV (5 J, 10 J, or 15 J/m2), and allowed to recover for 1 hr. Cells were then irradiated with IR (10Gy) and fixed 4 hours post IR. Fixed cells were coimmunostained with Abs to γ-H2AX and Rad51. Additional wt and heterozygous strains were also assayed (in panel b). (b) Additional BRCA1^{+/+} and BRCA1^{mut/+} strains were analyzed as described in (a). A graph depicting the fraction of cells in each additional HMEC strain that contain Rad51 foci after exposure to different doses of UV followed by 10Gy dose of IR, was plotted. The means results and standard deviations of data from at least three experiments are shown for each line. (c) Rad51 expression in BRCA1^{mut/+} and BRCA1+/+ HMEC lines. Whole cell extracts from various BRCA1^{mut/+} and BRCA1^{+/+} strains were analyzed by western blot. GAPDH was used as a loading control in these blots. (d) Combinations of BRCA1^{mut/+} and BRCA1^{+/+} HMECs (BRCA1^{+/+}/BRCA1^{+/+}=green, BRCA1^{mut/+}/BRCA1^{mut/+}=blue, and BRCA1^{mut/+}/BRCA1^{+/+}=red) were irradiated with different doses of UV (0 J/m², 3 J/m², 6 J/m², and 9 J/m²), allowed to recover for 1 hour, and then either treated with 0.2 uM PARP inhibitor (PI) olaparib (UV+PI) or with DMSO as control (UV). Cells were grown for 5 more days before harvesting for FACS analysis. Data is plotted for the three different cell combinations, and the error bars were calculated as the standard error propagation (SEP) in the ratios of each of the combinations in three independent experiments. Data marked with an asterisk (*) reveal statistically significant differences (P-value<0.05) between UV and UV+PI sets. (e) Model for BRCA1 mutation-driven tumorigenesis.

FIG. 7. Homogeneous Mass-Extend (hME) analysis and DNA sequencing to confirm BRCA1 mutations in fibroblasts and HMECs. (1A) The mutation present in each of the BRCA1^{mut/+} fibroblast lines, and one HMEC line (AR1) used in this study were confirmed by homogenous Mass-Extend (hME) analysis. hME profiles of a subset of the mutations is shown, and the rest are available upon request. Genotyping was performed by Sequenom MassARRAY technology (Sequenom Inc., San Diego, Calif.) using a locus-specific primer extension method, as previously described (MacConaill et al., 2009, Thomas et al., 2007). (1B, C) The mutation in each of the BRCA1^{mut/+} HMEC strains used in this study was confirmed by direct nucleotide sequencing (L). Corresponding WT sequences are shown on the Right.

FIG. 8. BRCA1 protein levels in BRCA1^{mut/+} and BRCA1^{+/+} cells, and FACS analysis to determine the cell lineage and replication status of BRCA1^{+/+} and BRCA1^{mut/+} cells. (a) Cell lineage for BRCA1^{+/+} and BRCA1^{mut/+} HMECs was determined by flow cytometry analysis of cell surface markers (EpCAM, CD24, CD49f and CD44). This analysis was carried out for the following HMEC strains BRCA1^{mut/+} (CP10, CP16, CP17, AR16, 79 and AR11) and BRCA1^{+/+} (CP22, CP29, CP32, AR7). (b) Nuclear extracts from BRCA1^{mut/+} and BRCA1^{+/+} strains were prepared and analyzed for BRCA1 protein level. A non-specific band and/or level of GAPDH were used as a loading control. The intense BRCA1 band in 47 (185delAG) is likely the previously discovered truncated product of this mutant allele⁵¹. (c) Replication profiles for each of the HMEC and fibroblast strains were assayed by BrDU based FACS analysis. Briefly, the cells were pulse-labeled with 10 uM BrdU for 30 minutes (for HMECs) and 1.5 hours (for fibroblasts-which proliferate more slowly than HMECs) and then fixed for FACS analysis.

FIG. 9. Satellite RNA induction and Slug expression in BRCA1 WT and mutant HMECs. (a, b) In situ RNA hybridization was carried out for HSATII in BRCA1^{+/+} (CP22, CP32, CP29) and BRCA1^{mut/+} (CP10, 79, CP16 and CP17) lines. Images for CP22 and CP10 are shown in the figure. SW620 is a colon cancer line and was used as a positive control for HSATII (it expresses HSATII after dox induction). GAPDH was used as a positive control for RNA FISH in these experiments. (c) Steady state levels of Slug in BRCA1^{+/+} HMECs (AR7) were similar to those BRCA1^{mut/+} strains (CP10, CP16 and CP17). MDA-MB-231 (a basal-like sporadic breast cancer cell line) was used as a positive control here. MCF7 (a luminal line) served as a negative control for SLUG expression. Each panel was taken from the same blot, but the top panel was exposed to vinculin Ab to yield loading control results. The bottom-most panel represents a longer exposure than the middle one. Both reflect SLUG protein abundance.

FIG. 10. Generation of ssDNA and pRPA32 loading on chromatin after stalled fork induced DNA damage. (a) phospho-RPA32 (pRPA32) loading on chromatin is BRCA1 dependent. U20S cells infected with lentiviral shRNA directed at BRCA1 (ShB) exhibit reduced pRPA32 loading, compared to control infected (ShRNA directed at Luciferase, ShL), after HU -induced stalled fork formation. (b, c, d) BRCA1^{mut/+} and BRCA1^{+/+} fibroblast and HMEC strains were either mock-treated or irradiated with 30 J/m² of UV and/or exposed to HU (10 mM for 3 hours). All were harvested 3 hours post damage. Chromatin-rich extracts were prepared and analyzed by western blot for the presence of pRPA32. Each panel represents a different blot. Strains depicted in a given blot replicated similarly on the day of the experiment. (e) BrdU assay for ssDNA generation after UV-induced stalled replication forks. BRCA1^{+/+} (1002) and BRCA1^{mut/+} (39 and 1075) fibroblast strains were irradiated with low dose UV (5 J/m2) and fixed 4 hrs later to detect the presence of ssDNA. Cells were immunostained for BrdU with or without HCl denaturation of DNA. Details of the protocol are provided in Materials and Methods. (f) Data analyzed in (e) is plotted. Upper panel/chart details percentage of BrdU positive cells in different fibroblast strains. Bottom panel/chart details average intensity of BrdU positive cells as determined by ImageJ software. Error bars represent standard deviation in three independent experiments.

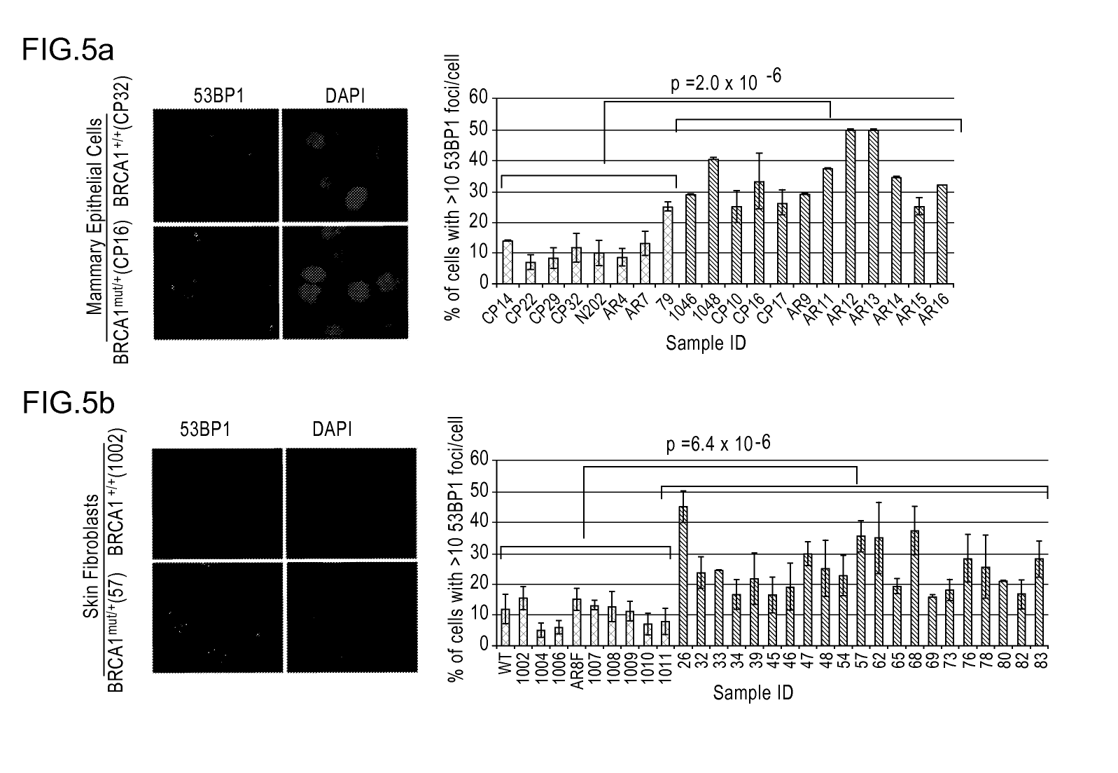

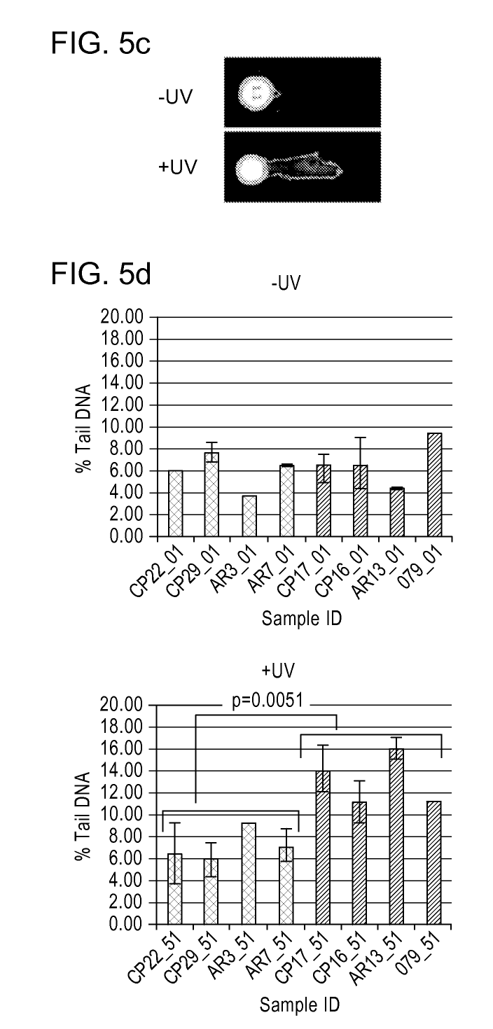

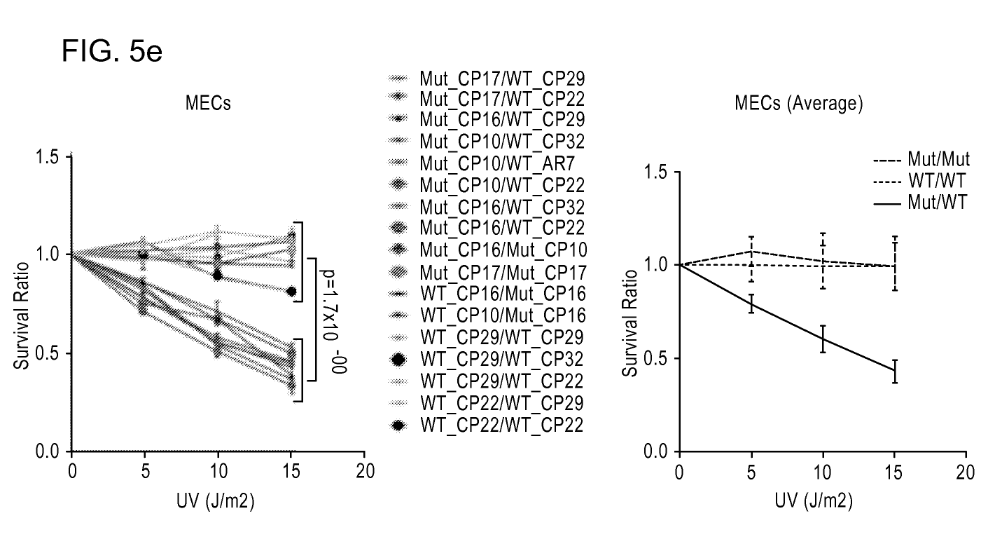

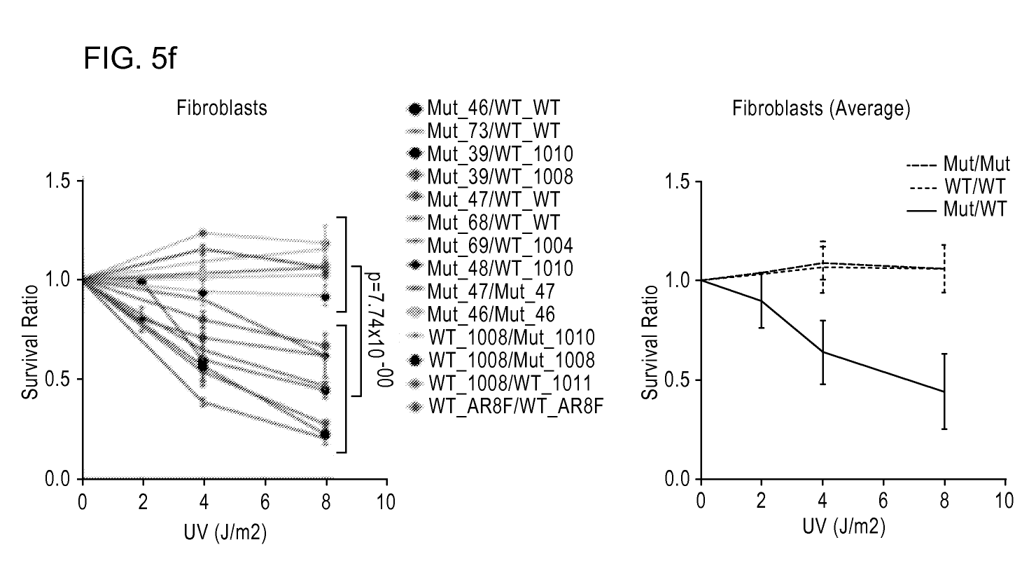

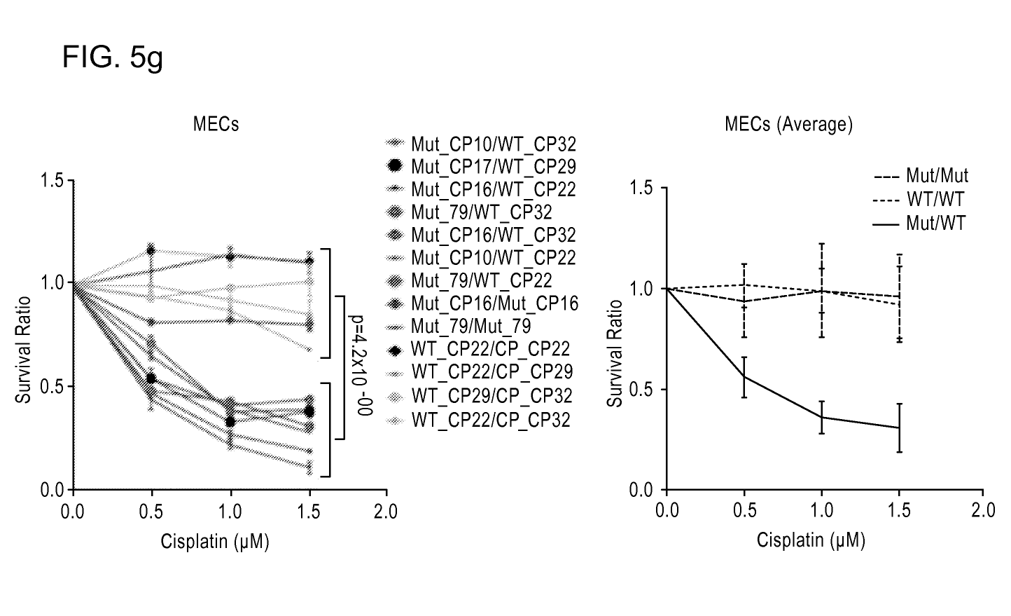

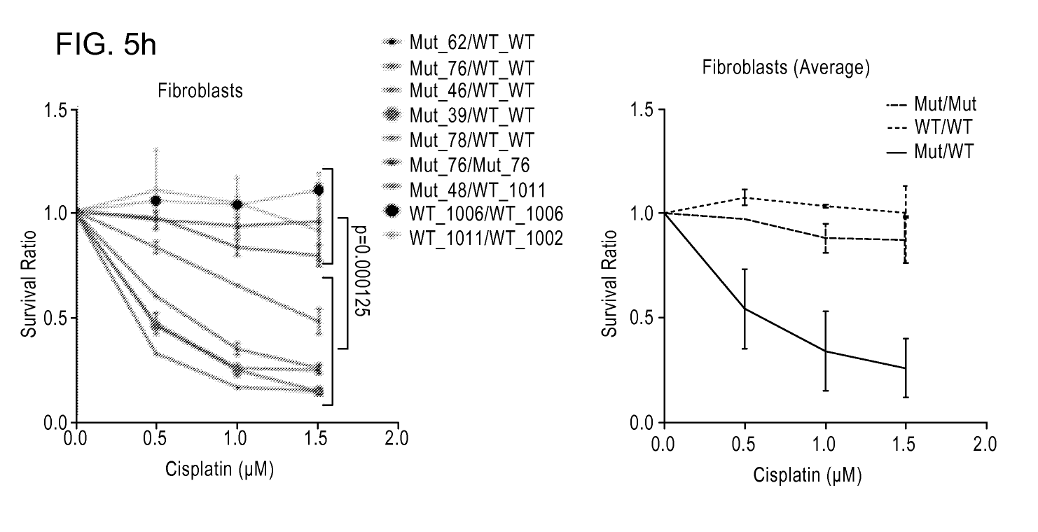

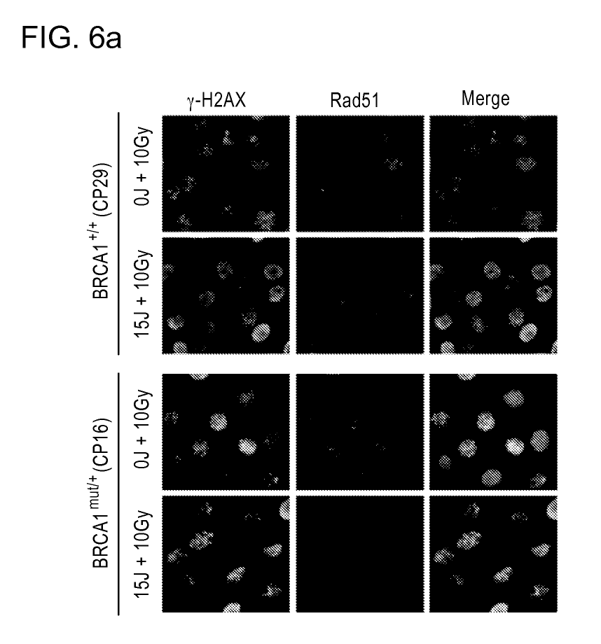

FIG. 11. The stability of stalled forks is compromised in BRCA1^{mut/+} cells. (a) BRCA1^{mut/+} (47 and 46) were infected with either eGFP expressing or HA-tagged BRCA1 lentiviral vector. Infected cells were grown in presence of Blasticidin (5 ug/ml, selection marker) for 5 days and then harvested to prepare whole cell lysates for immunoprecipitation (IP) with HA. Western blots for the IP samples were probed with antibody to BRCA1 (MS110). (b) CP16, CP17 and 79 (BRCA1^{mut/+}) cell lines were irradiated with either 15 J/m2 UV alone (UV) or with UV followed by 10gy dose of IR (UV+IR). Cells were harvested 4 hours post damage, and whole cell extracts wereprepared. These extracts were analyzed by western blotting for BRCA1. GAPDH served as a loading control in these experiments. (c) Distribution of IdU tract lengths, after incubation of cells in presence of HU and/or absence of HU, is plotted as a curve for BRCA1^{+/+} fibroblast strains (AR20L) and BRCA1^{mut/+} (46 and 39) strains. Experimental design is as described in FIG. 5c. (d) Heterozygous BRCA1^{mut/+} cells reveal a compromised DNA repair efficiency compared to WT BRCA1^{+/+} cells after exposure to low dose UV (5 J/m2). DNA damage was measured as a percentage of DNA in comet tails after UV-induced DNA damage. Representative images for comets in unirradiated (−UV) and irradiated (+UV) samples are shown. (e) BRCA1^{mut/+} and BRCA1^{+/+} HMECs were irradiated with 5 J/m2 of UV and allowed to recover for 3 hours before carrying out the alkaline comet assay. Percentage of DNA in the comet tails is plotted for unirradiated (left panel) and irradiated cells (right panel). Green bars=BRCA1^{+/+} and red=BRCA1^{mut/+} cells. The mean result and standard deviation of at least three different experiments is plotted. In each experiment at least 250 individual cells were scored for percentage of DNA in the comet tails using CellProfiler software.

FIG. 12. Recruitment of CtIP, Rad51 and Mre11 to sites of stalled forks (after UV-induced DNA damage) in BRCA1^{+/+} and BRCA1^{mut/+} strains. BRCA1^{+/+} and BRCA1^{mut/+} HMECs and fibroblast strains were irradiated with 30 J/m2 UV through micropore filters. Cells were fixed 3 hours post UV-induced DNA damage and immunostained for CtIP (a), Rad51 (b) and Mre11 (c). CPD (cyclobutane pyrimidine dimers) and γ-H2AX staining was used to mark the sites of UV damage/stalled forks. Plots on the right show percentage of cells with the respective proteins (CtIP, Rad51 and/or Mre11) localized in micropores. Green bars denote BRCA1^{+/+} strains and red bars denote BRCA1^{mut/+} strains in all the plots.

### DETAILED DESCRIPTION OF THE INVENTION

The invention is based in part in the discovery that normal tissues from individuals with the known cancer-causing truncating BRCA1 mutation (BRCA1^{mut/+} cells) are defective in stalled fork replication repair (SFR) and in the suppression of fork collapse, i.e. replication stress.

BRCA1 is a tumor suppressor gene, and germ line BRCA1 mutations increase greatly the risk of breast and ovarian cancer. While all cells of males and females with germline BRCA1 mutations exhibit a heterozygous BRCA1^{mut/+} genotype, cancer develops primarily in females, often at young ages and affects almost exclusively the breast and ovaries. Why BRCA1 is largely a breast and ovarian cancer susceptibility gene, why males are largely protected from BRCA1 cancer, and how an ostensibly normal epithelial cell in a BRCA1 mutation carrier (BRCA1^{mut/+}) gives rise to proliferating and invasive tumor cells are largely unknown.

A BRCA1 loss of heterozygosity (LOH) event is a consistent characteristic of fully developed BRCA1-linked tumor cells. Two generic models describe the chain of events that precede it and the emergence of overtly neoplastic mammary epithelial cells (HMECs). In one, HMECs, despite being heterozygous, are histologically and biologically normal prior to the emergence of LOH. They fail to exhibit a significant defect in BRCA1 function. Here key events that transform a cell to malignancy follow the loss of all BRCA1 function at the LOH event and are often preceded by acquisition of a p53 mutation which sustains cell viability in the face of emerging genome disorder.

In the other model BRCA1^{mut/+} HMECs are haploinsufficient for the performance of one or more BRCA1 functions even before any signs of a neoplastic cell phenotype emerge. This model implies that, from the time that mammary epithelial development is complete or at some relatively early time thereafter, BRCA1^{mut/+} HMECs cannot perform all BRCA1 genome integrity maintenance functions at normal amplitude. These abnormalities may increase the likelihood that early steps in a mammary tumorigenesis process begin, though they may only become clinically apparent years later.

In this regard, there is growing evidence of a defect in normal mammary epithelial progenitor differentiation in histologically normal, BRCA1 heterozygous mammary tissue, implying that the second model is more likely valid than the first. Thus, determining whether BRCA1 heterozygosity confers haploinsufficiency upon HMECs for any of the multiple, known, BRCA1 functions is a potentially valuable step in achieving a better understanding of BRCA1 mutation-driven cancer predisposition. Thus, the inventors have analyzed a new collection of primary mammary BRCA1^{mut/+} epithelial cells and skin fibroblasts obtained from BRCA1 mutation carriers for such functions.

All BRCA1 heterozygous cells exhibited multiple, normal BRCA1 functions, including the support of homologous recombination-type double strand break repair (HR-DSBR), cell cycle-associated checkpoint functions, centrosome number control, spindle pole formation, Slug expression and satellite RNA suppression. By contrast, nearly all cells were defective in the repair of stalled replication forks (SFR) and in the suppression of fork collapse, i.e. replication stress. These defects were rescued by reconstituting BRCA1 heterozygous cells with wild-type BRCA1 cDNA, indicating that they are a product of BRCA1 haploinsufficiency. In addition, the development of sufficient replication stalling rendered BRCA1^{mut/+} cells defective in an otherwise intact BRCA1 function, HR-DSBR. No such ‘conditional’ haploinsufficiency was detected in any of the other non-haploinsufficient functions, noted above. Given the importance of replication stress in epithelial cancer development and of an HR defect in breast cancer pathogenesis, these defects, when they develop serially, could contribute to the BRCA1 breast cancer development pathway.

These results suggest that defective SFR could be one of the early events that trigger tumorigenic events in cells of BRCA 1 mutation bearing individuals. Thus, determining if an individual having a BRAC1 mutation has defective SFR may predict break cancer risk for the individual.

Accordingly, the present invention provides a method of rapidly and reliably testing individuals with known cancer associated germline mutations such as BRCA1 and BRCA2 mutations for susceptibility for SFR. The method comprises co-culturing labeled cells from patients with germline mutations in known cancer-associated genes and labeled wildtype control cells; exposing the cells to a DNA damaging agent and determining the relative abundance of each cell population. When the ratio of patient cells to control cells is less than one (1) then the individual is at an increased risk for developing breast cancer.

By identifying individuals at a greater risk of developing breast cancer allows for the prophylactic treatment recommendation to be made for the individual.

The relative abundance of the cell population can be determined by any method known on the art, such as fluorescence activated cell sorting (FACS). FACS is used to sort individual cells on the basis of optical properties, including fluorescence. It is generally fast, and can result in screening large populations of cells in a relatively short period of time.

The term “flow cytometer” as used herein refers to any device that will irradiate a particle suspended in a fluid medium with light at a first wavelength, and is capable of detecting a light at the same or a different wavelength, wherein the detected light indicates the presence of a cell or an indicator therein. The “flow cytometer” may be coupled to a cell sorter that is capable of isolating the particle or cell from other particles or cells not emitting the second light. Preferred cell types for use in the invention are cells capable of mitosis . Suitable cells include, but are not limited to, mammalian cells, including animal, primates, and human cells. Preferably the cells are fibroblasts such as skin cells.

By labeled cells is meant that the cells are labeled with a detectable label that allows the two populations to be distinguished from one another.

The detectable label is for example a dye. A dye (generally a fluorescent dye as outlined below) is introduced to cells and taken up by the cells. Once taken up, the dye is trapped in the cell, and does not diffuse out. As the cell population divides, the dye is proportionally diluted. That is, after the introduction of the inclusion dye, the cells are allowed to incubate for some period of time.

The dye can passively enter the cells, but once taken up, it is modified such that it cannot diffuse out of the cells. For example, enzymatic modification of the dye may render it charged, and thus unable to diffuse out of the cells. For example, the Molecular Probes CellTracker™. dyes are fluorescent chloromethyl derivatives that freely diffuse into cells, and then glutathione S-transferase-mediated reaction produces membrane impermeant dyes.

Suitable dyes include, but are not limited to, the Molecular Probes line of CellTracker™ dyes, including, but not limited to CellTracker™ Blue, CellTracker™ Yellow-Green, CellTracker™ Green, CellTracker™ Orange, PKH26 (Sigma), and others known in the art; see the Molecular Probes Handbook, supra; chapter 15 in particular.

Other suitable dyes include, the Molecular Probes line of CellTrace™ CFSE Cell Proliferation Kit (e.g. CellTrace™ CFSE Cell Proliferation Kit, for flow cytometry).

In general, dyes are provided to the cells at a concentration ranging from about 100 ng/ml to about 5 μg/ml, with from about 500 ng/ml to about 1 μg/ml. A wash step may or may not be used.

The cells and the dye are incubated for some period of time, to allow cell division and thus dye dilution. The length of time will depend on the cell cycle time for the particular cells; in general, at least about 2 cell divisions are preferred, with at least about 3 being particularly preferred and at least about 4 being especially preferred. The cells are then sorted as outlined below, to create populations of cells that are replicating and those that are not.

Relative abundance of the particular cell type (i.e., patient or control) is determined by measuring the fluorescence in different cell populations, and comparing the determinations to one another.

Cells used in the present invention may also have been previously cultured in vitro or ex vivo (such as by use of tissue culture medium) prior to being used in the methods of the invention. The culture method or means may be any known or accepted in the art, so long as they are suitable to maintain or improve the viability of at least a portion of the cells being cultured. While any suitable media may be used, preferred media would have reduced amounts of, or the absence of, agents which interfere with the conversion of a pre-dye to a detectable dye within a viable cell. Non-limiting examples of such an agent include antioxidants and phenol red, which is preferably omitted from culture media, such as those based on Hank's Balanced Salt Solution or Dulbecco's Modified Essential Medium (DMEM), used in the practice of the present invention. Of course culturing may be by use of any suitable device, including incubators, and chamber.

Reference herein to a “population of cells” means two or more cells. A “substantially homogenous population” means a population comprising substantially of only one cell type. A “cell type” means a population of cells which are distinguished from other cells by a particular common characteristic. Preferably, the substantially homogenous population comprises a population of cells of which at least about 50% are of the same type, or at least about 60%, or at least about 70%, or at least about 80%, or at least about 90%, or at least about 95% or above such as at least about 100% are of the same type.

The term “subject” or “patient” refers to any human or nonhuman organism.

A “control subject” or a control sample refers to any human or nonhuman organism or sample derived therefrom that does not have a known cancer associated germline mutations.

The term “biological sample” may include any sample comprising biological material obtained from e.g. an organism, body fluid, waste product, cell or part of a cell thereof, cell line, biopsy, or tissue culture.

The term DNA damaging agent includes replication-stalling agents. DNA damaging agents include for example cisplatin, hydroxyurea and UV-C (ultraviolet radiation)

### EXAMPLES

### Example 1

### General Methods

Cell Culture, and HMEC and Fibroblast Isolation From Tissue Biopsies

Tissue samples were briefly washed in PBS and then minced and digested overnight in medium containing 1 mg/ml of collagenase type III (Roche). For digestion, MEGM medium (Lonza) was used for breast tissue, and Dulbecco's modified Eagle's Medium (DMEM) with 5% fetal bovine serum (FBS) for skin tissue. The digested tissue was pelleted and fibroblasts were cultured in DMEM supplemented with 15% FBS (Gibco), 1% Pen/Strep (Gibco) and 1% Glutamine (Gibco), and HMECs were grown in MEGM medium supplemented with 1% Pen/Strep.

Transfection, Infection and Selection

For siRNA experiments, cells were grown in 6-well plates and transfected with 100 pmoles of siRNA with RNAiMAX (Invitrogen) according to the manufacturer's protocol. Where relevant, experiments were initiated 48 hours after transfection. All siRNA oligonucleotides were purchased from Thermo Scientific. siRNA oligonucleotides used were: siBRCA1 (On Target Plus BRCA1, catalog number CTM-41735), and siGAPDH (On Target Plus GAPDH, catalog number D-001830-01-20).

For shRNA experiments, shRNA encoding lentiviruses were generated using 293FT packaging cells in the presence of lipofectamine (Invitrogen). Cells infected with lentiviruses were selected transiently using 2.5 μg/ml puromycin (Santa Cruz). ShBRCA1 and shLuc were acquired from The RNAi Consortium (TRC). The target sequence for shBRCA1 was AGAATCCTAGAGATACTGAA. For BRCA1 reconstitution experiments, lentiviral packaging plasmids VSVG and PSPAX were used to package BRCA1 and/or eGFP plasmid in 293FT cells using lipofectamine (Invitrogen). Cells were infected with the lentivirus and selected using 6 μg/ml of Blasticidin (Invitrogen). For color-coding experiments, hTert and GFPhtert containing retroviruses were prepared by packaging the plasmids pMIG-hTERT and pBABE-hygro-hTERT with retrovirus packaging plasmids pMD-MLV and pMD-G in 293FT cell line. hTERT infected cells were selected with hygromycin B (Roche) (50 μg/mL).

Cell Extracts, Immnoblotting, and Antibodies

Whole cell extracts were prepared by lysing the cells in NETN300 lysis buffer (300 mM NaCL, 20mM Tris-HCl buffer pH 7.8, 0.5% NP-40, 1 mM EDTA) for 1 hour at 4° C. Nuclear extracts were prepared by pre-extracting the cytoplasmic protein fraction by incubating the cells in pre-extraction buffer i.e PEB (0.5% Triton-X-100, 20 mM HEPES, 100 mM NaCl, 3 mM MgCl2 and 300 mM Sucrose). Incubation was carried out at 4° C. for 20 minutes. Cells were pelleted, washed once in PEB, and lysed in NETN 400 lysis buffer (400 mM NaCL, 20 mM Tris-HCl buffer pH 7.8, 0.5% NP-40, 1 mM EDTA) for 45minutes at 4° C. All the lysis buffers were supplemented with 1× protease inhibitor (Roche) and Halt Phosphatase inhibitor (Thermo Scientific). Chromatin extracts were prepared as described previously⁴³ Immunoprecipitation for HA-tagged BRCA1 was carried out by incubating whole cell extracts with HA antibody (Covance) for 2 hours, followed by 1 hour incubation with Protein A beads (GE healthcare) at 4° C. The beads were washed in NETN 150 buffer (150 mM NaCL, 20 mM Tris-HCl buffer pH 7.8, 0.5% NP-40, 1 mM EDTA). Antibodies used for western blotting were phospho-RPA32 (Bethyl Labs; 1:2000), BRCA1 (SD118; 1:1000), GAPDH (Santa Cruz; 1:4000), pS53BP1-S25 (Novus Biologicals; 1:5000), Rad51 (Santa Cruz; 1:600), Slug (Cell Signaling; 1:3000), Vinculin (Santa Cruz; 1:1000), BRCA1 (MS110; 1:1000), HA (Covance; 1:4000).

Immunofluorescence and Antibodies

Cells on coverslips were fixed with 4% paraformaldehyde/2% Sucrose for 15 minutes, and triton extracted (0.5% Triton X-100 in PBS) for 4 minutes. Cells were blocked with 5% BSA/PBST and then incubated with respective antibodies for 30 minutes at 37° C. followed by incubation with secondary antibodies (FITC or Rhodamine) for 30 minutes at 37° C. Primary antibodies used in immunofluorescence studies were: BRCA1 (Upstate; 1:500), phospho-53BP1(S1778) (Cell Signaling; 1:200), RPA (Cal Biochem; 1:100), 53BP1 (Bethyl Labs; 1:2000), Rad51(Santa Cruz; 1:150), HA (Covance; 1:500), and γ-H2AX (Millipore; 1:5000). For TPX2 (Bethyl Labs; 1:400) and γ-tubulin (Sigma-Aldrich; 1:1000) staining, the cells were pre-fixed with acetone:methanol (3:7) at −20° C. for 10 minutes, followed by triton extraction (0.2% triton-X-100 in 20mM HEPES, pH 7.4, 50 mM NaCl, 3 mM MgCl2, 300 mM Sucrose) at room temperature. Primary and secondary antibody staining was carried out as described above.

Cell Treatments

For analysis of phospho-RPA32 loading on chromatin, cells were treated with HU (Sigma) and/or UV. Cells were incubated in HU (10 mM) containing medium for 4 hours before harvesting for further analysis. For UV treatment, cells were irradiated with 30 J/m² UV with a 254 nm UV-C lamp (UVP Inc, Upland, Calif.). Cells were harvested 4 hours post UV. UV-irradiation through a micropore membrane was done as described previously⁴³. For color-coded FACS based cell survival assay, Parp inhibitor olaparib (Selleck) was added to the final concentrations of 0.2 μM, 0.4 μM and 0.6 μM for 6 days; cisplatin (Novaplus) was added to the final concentrations of 0.5 μM, 1.0 μM and 1.5 μM for 24 hours. Medium was replaced and the cells allowed for grow for 5 more days. Different doses of UV used were 5 J/m², 10 J/m² and 15 J/m², cells were allowed to recover for 6 days before harvesting them for FACS analysis. Laser induced DNA-breaks were generated as described in Greenberg et al¹¹.

Sequencing and hME (homogeneous Mass-Extend)

Cells lines were sequenced to confirm their mutations via direct sequencing or by homogeneous Mass-Extend sequencing method. Genomic DNA was prepared using Blood and DNeasy kit (Qiagen), and a mutation locus specific PCR reaction was carried out to amplify the region of interest. For direct sequencing, the amplified PCR products were purified using Qiagen's PCR purification kit, and were sent for sequencing. For hME analysis, a locus-specific primer extension of the PCR amplified region is carried out in presence of a mixture of di-deoxy and deoxy NTPs. Allele-specific extension products are analyzed by mass spectrometry to determine the specific sequence.

Comet Assay and Analysis

For detection of DNA breaks, alkaline comet assays were performed using the Single-Cell Gel Electrophoresis Assay kit (Trevigen) according to the manufacturer's instructions. The quantification of percentage of tail DNA was carried out using CellProfiler software.

Flow Cytometry, Checkpoints, and Color-Coding Based Cell Survival FACS Assay

For cell cycle analysis, cells were pulse-labeled with 10 μM BrdU for 30 minutes (for HMECs) and 1.5 hours (for fibroblasts) in respective culture medium. Single cell suspensions were fixed in 70% ice-cold ethanol. Cells were incubated with anti-BrdU FITC conjugate antibody (Becton Dickinson, 1:10 dilution made in Blocking solution from Thermo Scientific) at room temperature in dark for 45 minutes. Finally, the cells were resuspended in propidium iodide and RNAse staining buffer (Becton and Dickinson) and analyzed with Becton Dickinson FACS machine (Mountain View, Calif.).

For checkpoint assays, cells were irradiated with either UV and/or IR and allowed to recover for 2 hours. For S-phase checkpoint analysis, cells were incubated with BrdU as described above before harvesting and fixing for FACS analysis. For G2 checkpoint, fixed cells were incubated with Alexa Flour anti-phospho-histone H3 (Ser10) antibody diluted in 2% BSA/PBS at room temperature in dark for 2 hours. Cells were washed, and resuspended in propidium iodide and RNAse containing staining buffer.

For color-coded FACS based assay, GFP positive and GFP negative cells were mixed in equal numbers (8000 cells/strain) and plated in 6 cm² plates. After drug and/or UV treatment, cells were allowed to recover for 6 days before harvesting them for FACS analysis.

Satellite RNA q-RT-PCR

Cells grown in 6 cm2 plates were collected, RNA was prepared using RNeasy Plus Mini Kit (Qiagen), followed by cDNA preparation. q-RT-PCR was carried out with primers for SatA, SatIII, mcbox and β-Actin. More details and primer sequences are as described in Zhu, Q. et al²⁹.

### Example 2

### Primary Cell Isolation, Genotyping, and Lineage Determination

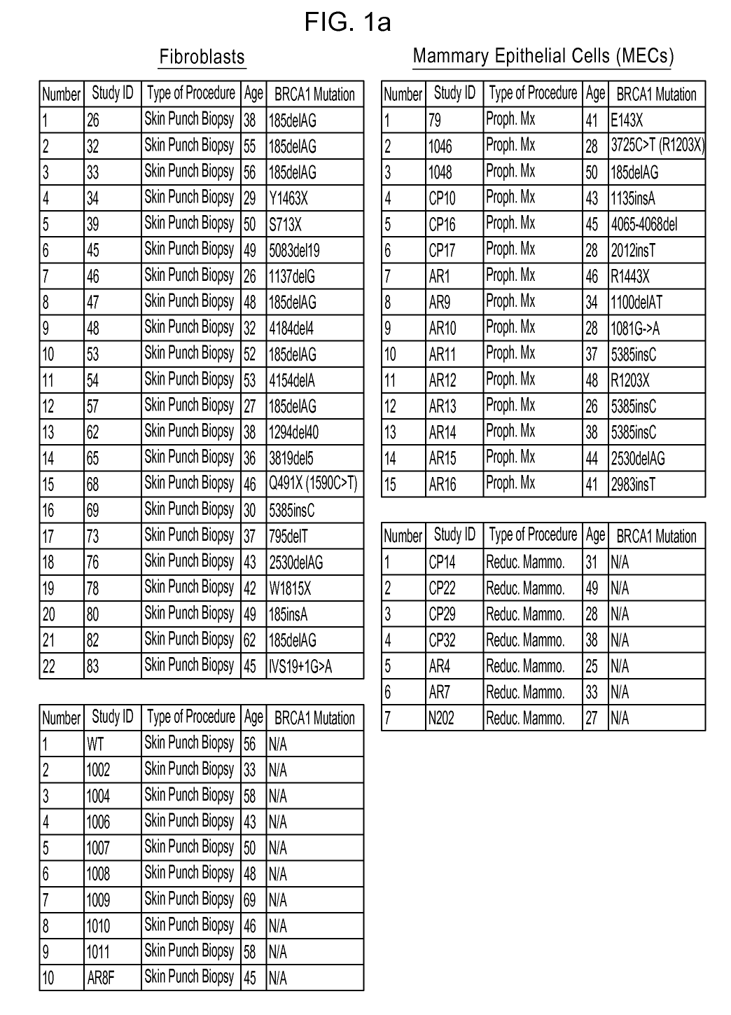

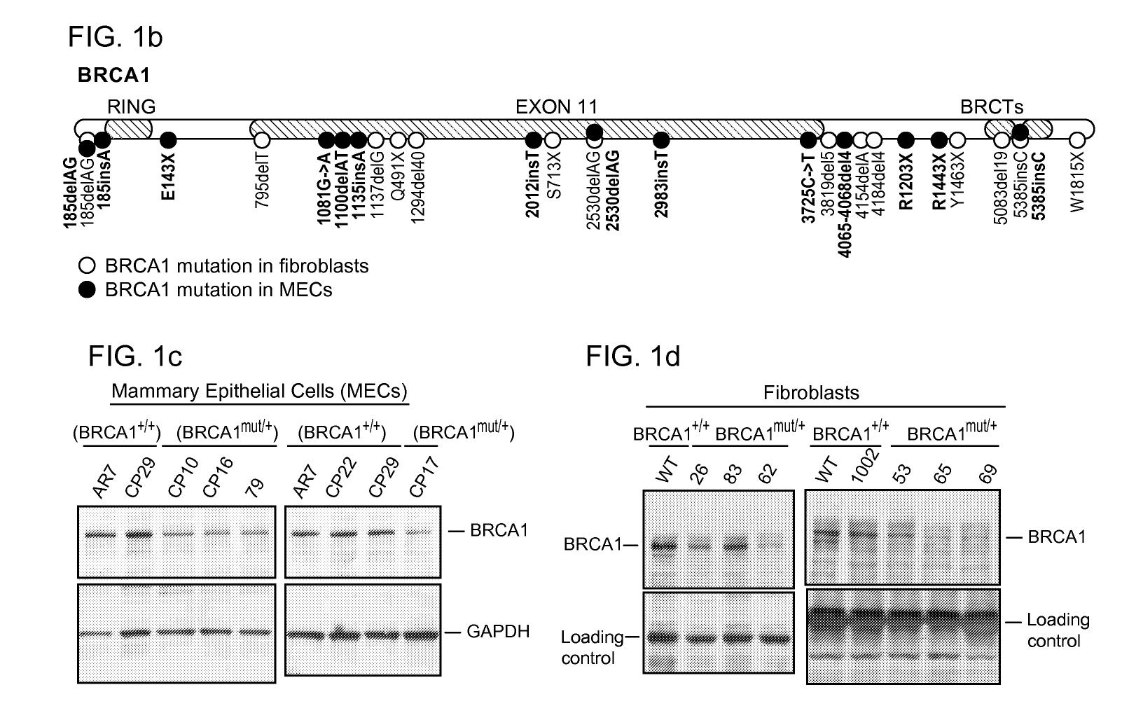

Established elements of BRCA1 function were analyzed in freshly isolated, morphologically non-neoplastic, primary human mammary epithelial cells (HMECs) and skin fibroblasts derived from multiple BRCA1^{+/+} and BRCA1^{mut/+} tumor-free women. These cells were collected under an IRB-approved protocol. 22 primary BRCA1^{mut/+} fibroblast cultures were derived from skin punch biopsies, and 15 primary BRCA1^{mut+} HMEC cultures were generated from prophylactic mastectomy samples (FIG. 1a). All BRCA1^{mut/+} volunteers were members of established, BRCA1 mutation-carrying families. No tumor tissue was detected in any of these samples. HMECs were cultured in serum-free media.

The properties of BRCA1^{mut/+} HMECs were compared with BRCA1^{+/+} HMECs (N=7), freshly derived from reduction mammoplasty tissue, and those of BRCA1^{+/+} skin fibroblasts with BRCA1^{+/+} skin fibroblasts (N=10; FIG. 1a). Mutations in all BRCA1 mutant fibroblasts, and one HMEC strain (AR1) were confirmed by homogenous Mass-Extend (hME) analysis^{22,23} (FIG. 7A), and in all other HMEC strains by direct BRCA1 gene sequencing (FIG. 7B, C). Together, this collection of BRCA1^{mut/+} mutations spans nearly the entire BRCA1 genome (FIG. 1b). determine the lineage of cells that grew out of our primary tissue samples under the culturing conditions used (details in Materials and Methods), we carried out flow cytometry (FACS)-based analysis of lineage markers (CD44, CD49f, CD24 and EpCAM). In this study, our primary BRCA1^{mut/+} and BRCA1^{+/+} HMEC cultures, were similarly enriched in early basal (CD44^{high}, CD24^{low}, CD49f^{high} EpCAM^{low}) as opposed to luminal progenitor cells (CD44^{low}, CD49f^{low}, CD24^{high}, EpCAM^{high})^{16,24} (examples are shown in FIG. 8a). For this analysis MCF7 was used as a luminal cell line control and MCFDCIS.com as a basal cell line control.

Furthermore, western blot analysis of whole cell extracts (for HMECs) and nuclear extracts (for fibroblasts) revealed that full length BRCA1 (i.e. p220) expression in BRCA1^{mut/+} HMEC (FIG. 1c) and fibroblast strains (FIG. 1d and FIG. 8b) was lower than that detected in wt BRCA1^{+/+} lines. This was in keeping with the proven genetic heterozygosity in these cells. Since BRCA1 is much more abundant in S and G2 than in G1, we only compared wt and heterozygous HMEC and fibroblast cultures that exhibited identical cell cycle FACS profiles (cf examples in FIG. 8c).

### Example 3

### Non DNA Repair-Driven BRCA1 Genome Integrity Functions

BRCA1 exhibits two types of genome integrity maintenance functions those that are directed towards the repair of DNA damage and checkpoint control, and others that sustain genome integrity by contributing to homeostatic functions that are not necessarily driven by DNA damage. In this context, we asked whether the lower expression of BRCA1 in BRCA1^{mut/+} than in wt cell cultures was associated with a deficiency in the latter BRCA1 functions. BRCA1 is required for the maintenance of centrosome number²⁵, mitotic spindle pole formation²⁶⁻²⁸, mammary development through the regulation of master genes like Slug¹⁹ , and heterochromatin-based satellite RNA suppression²⁹.

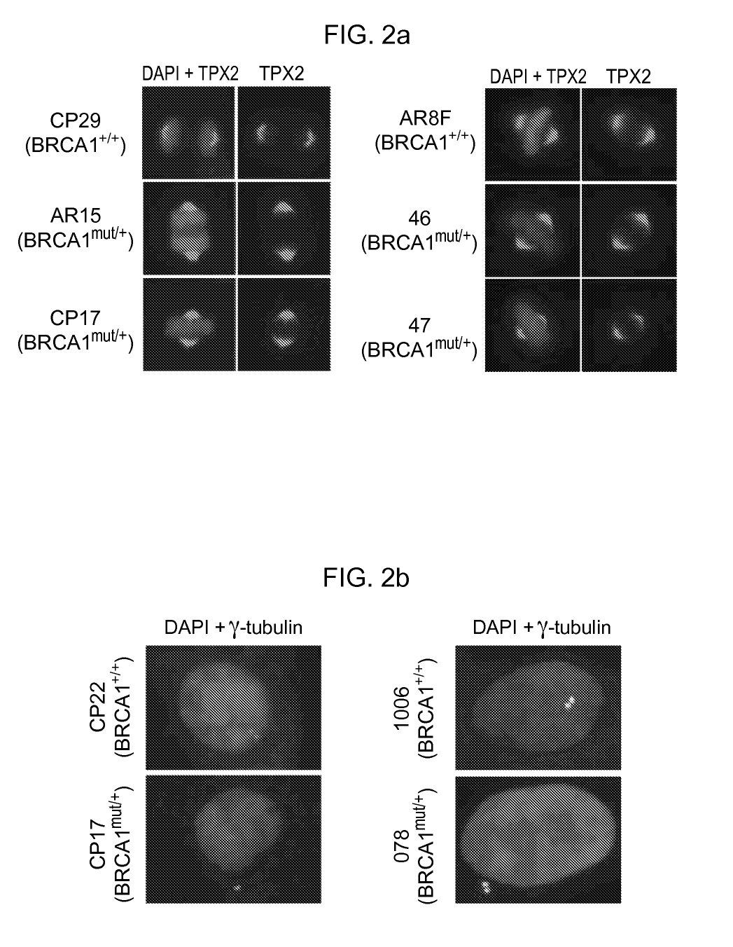

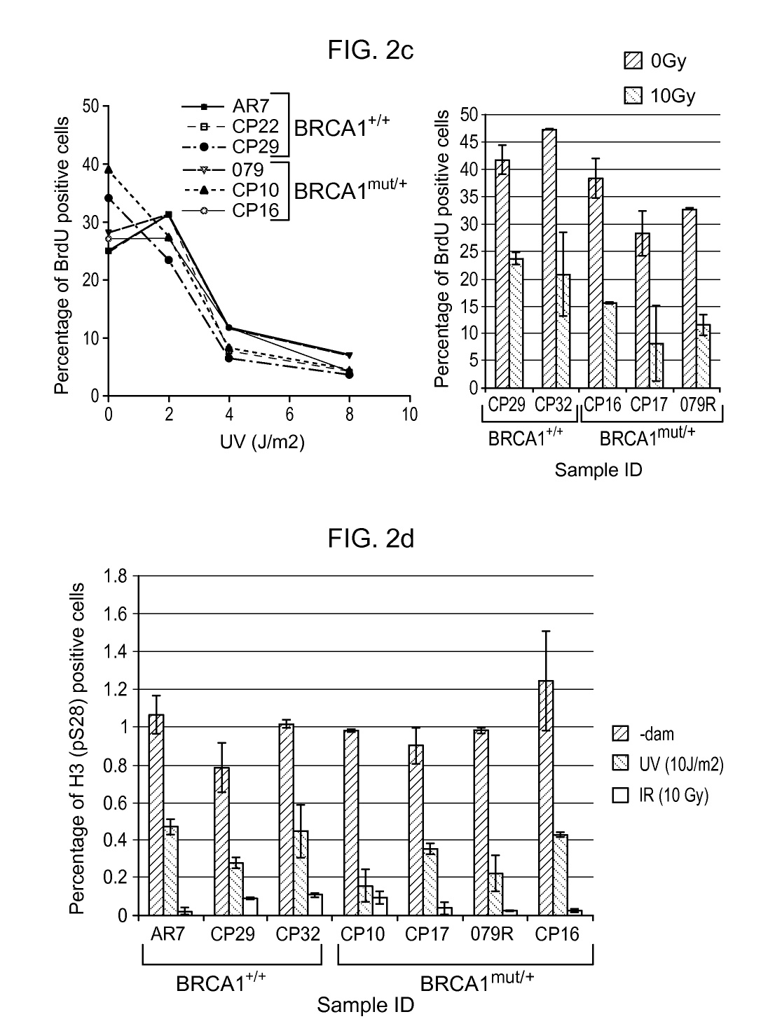

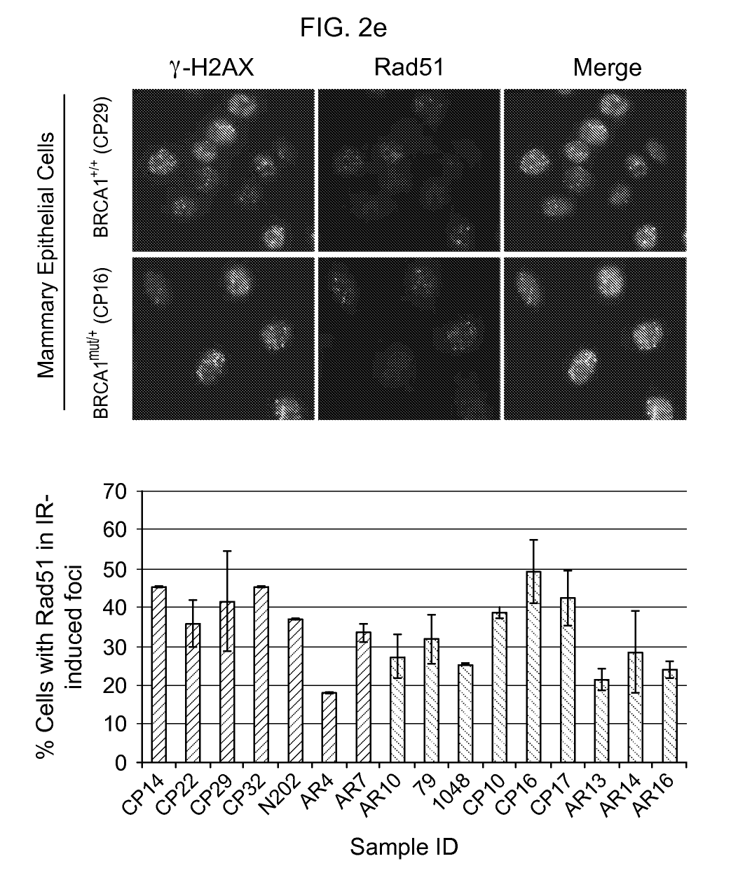

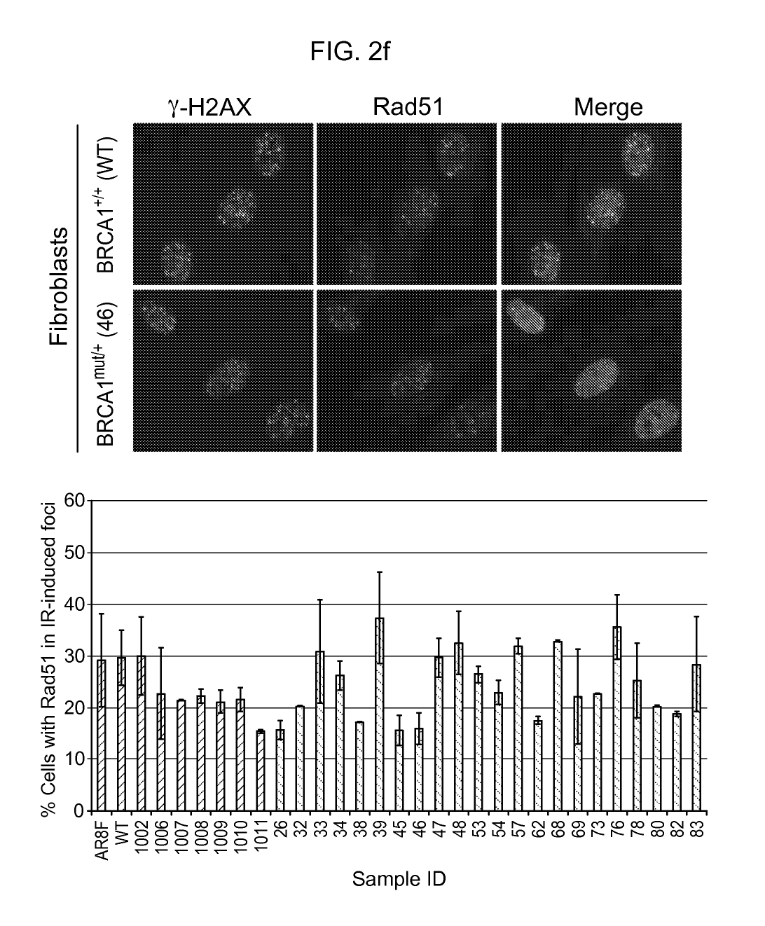

Each of these functions was compared in heterozygous (BRCA1^{mut/+}) and control (BRCA1^{+/+}) HMECs and fibroblasts. Spindle poles and spindle formation were analyzed by staining mitotic cells with antibody to TPX2. No abnormal spindle formation was detected in BRCA1^{mut/+} cells compared to wt BRCA1^{+/+} HMEC and fibroblast counterparts (FIG. 2a; and table S1). The effects of BRCA1 depletion on this function have been documented²⁶.

Similarly, centrosome number in individual primary cells was tested by staining with antibody to γ-tubulin (FIG. 2b). We found that none of the BRCA1^{mut/+} and BRCA1^{+/+} primary cell cultures (HMECs and fibroblasts) contained greater than 2 centrosomes, implying that centrosome maintenance was normal in these different BRCA1^{mut/+} strains (FIG. 2b). Although we did not detect any evidence of centrosome amplification in multiple BRCA1 heterozygous cells, work from the Polyak group¹⁵ with BRCA1 heterozygous tissue has previously observed a small increase of centrosome amplification (˜5%) in cells in heterozygous mammary tissue compared to 2.5% in wt tissue.

De-repression of satellite RNA transcription is also a feature of BRCA1 mutant tumors²⁹. To test whether this phenotype was present in heterozygous BRCA1 HMECs, two approaches were employed. Quantitative RT-PCR (q-RT-PCR) was performed using primers directed against alpha satellite variants (SATIII, SATa and mcBox), and satellite RNA transcript levels were also estimated by RNA FISH directed at another satellite RNA, HSATII. Very low levels of satellite RNA were present in primary HMECs, making it difficult to detect any satellite RNA signal by RNA FISH (FIGS. 9a and b). The colon cancer line, SW620, which expresses abundant, readily detectable HSATII upon addition of doxycycline, served as a positive control. Analysis by q-RT PCR did not consistently reveal major differences in the level of satellite RNA transcripts in multiple heterozygous HMECs when compared to wt controls. Thus, the insensitivity of the assay made it difficult to determine whether or not consistent differences in satellite RNA abundance exist.

To address the effect of BRCA1 heterozygosity on Slug expression¹⁹, we compared the level of Slug by western blot analysis in BRCA1^{+/+} and BRCA1^{mut/+} HMECs. In these experiments MCF7 (a luminal line) was used as a negative control and MDA-MB-231 (a basal line) was used as a positive control. No reproducible difference in Slug expression was detected between the BRCA1^{+/+} (AR7) and BRCA1^{mut/+} (CP10, CP16 and CP17) strains that were tested FIG. 9c).

### Example 4

### DNA Damage Checkpoints

BRCA1 plays an important role in regulating both the S phase and G2 checkpoints after DNA damage^{30,31}. The efficiency of post-damage checkpoint activation was also tested in BRCA1 heterozygous cells. We were unable to detect any significant difference in the ability of BRCA1^{+/+} and ^{mut/+} lines to mount either an S phase (FIG. 2c, left and right panel) or a G2 checkpoint response (FIG. 2d) following IR or UV induced DNA damage.

### Example 5

### BRCA1 DNA Repair Functions-Double Strand Break Repair

BRCA1 plays an essential role in HR-DSBR^{32,33}. When HR-DSBR is intact, DSBs are repaired in an error-free manner. Moreover, defective HR-DSBR is a well-known property of BRCA1 and related, inherited breast cancers; and molecular epidemiology results suggest that it is a risk factor for these cancers^{2,6,7}.

In response to DSB, BRCA1 is attracted to discrete sites of DSB-containing damage, where it directs a complex HR repair response^{12,34}. Long-standing results show that in BRCA1^{+/-} ES cells³⁵ HR function is normal until both copies of BRCA1 are inactivated (BRCA1^{-/-}). By contrast, others have reported that targeting one copy of BRCA1 with a mutation (e.g 185delAG) in an established, spontaneously immortal line of human HMECs resulted in a subtle HR defect³⁶. Thus, a detailed analysis of multiple, primary human BRCA1^{mut/+} and BRCA1^{+/+} HMECs and fibroblasts was undertaken to search for evidence of BRCA1 haploinsufficiency for HR-DSBR in this setting.

Two, well-validated assays were set up to measure the HR-DSBR competence of these cells first by testing the recruitment of Rad51 (an indicator of ongoing HR) to sites of DSBs, and second by measuring the sensitivity to PARP inhibitors (PI). The first assay clearly showed that BRCA1^{mut/+} HMECs and fibroblasts were as competent as BRCA1^{+/+} cells in recruiting Rad51 to sites of DSBs (FIG. 2e, 2f). Second, like HR DSBR-competent cells, they were also insensitive to olaparib (a PARP inhibitor). This assay, described below, relies on the observation that sensitivity to PARP inhibitors (PI) is dependent upon the existence of an HR defect. Indeed, BRCA1 tumor lines (which lack functional BRCA1 and reveal a defect in HR) are more sensitive to these agents than BRCA1^{+/+} cells³⁷⁻³⁹.

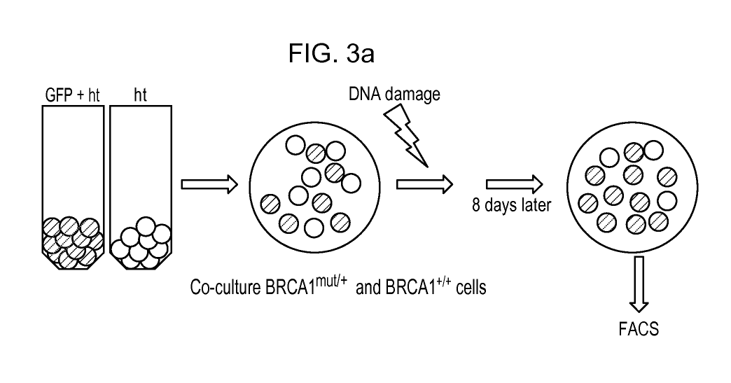

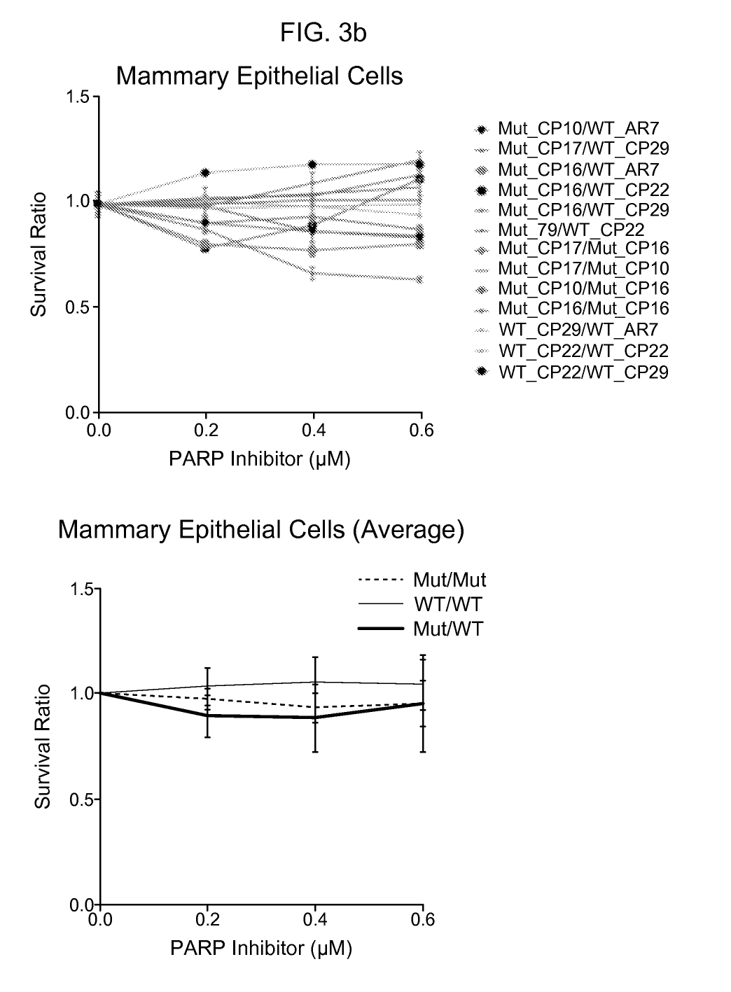

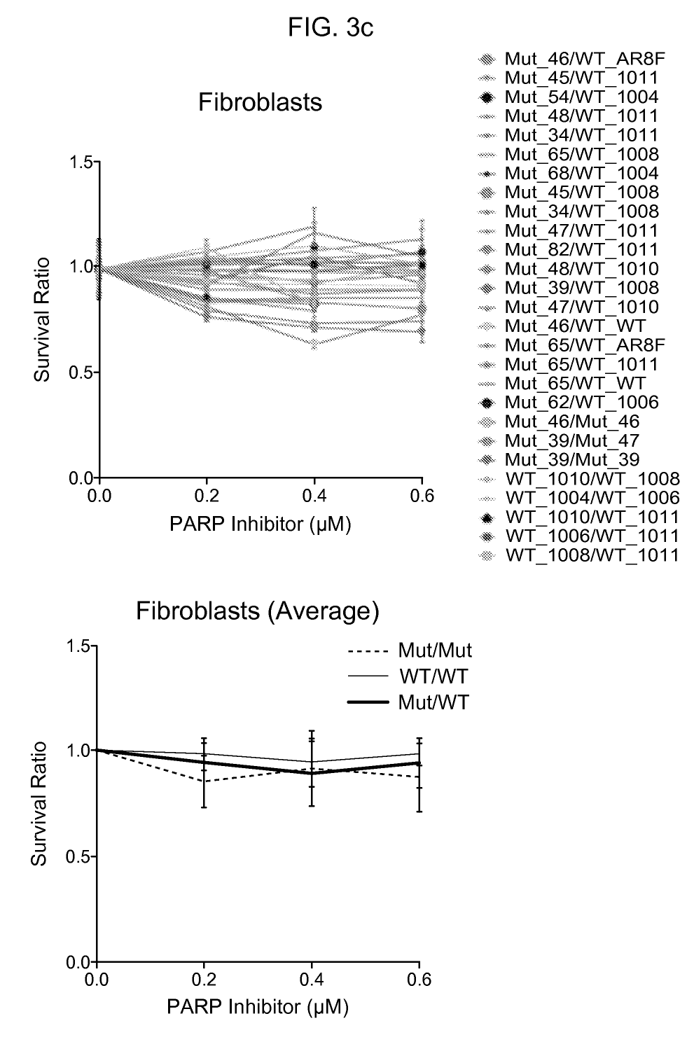

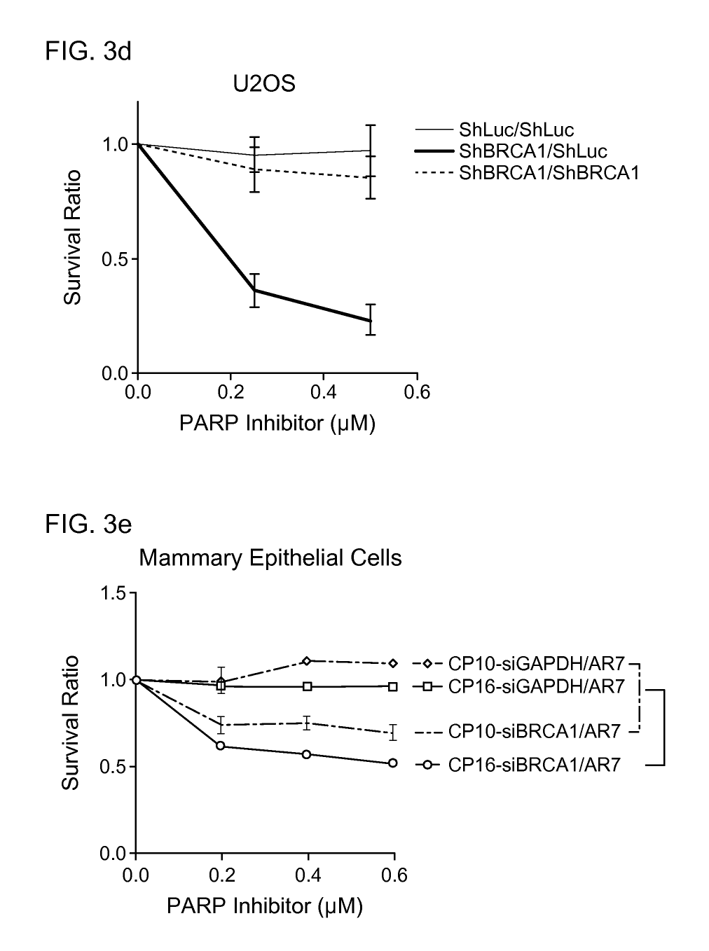

To study the effect of PARP inhibitors in our collection of BRCA1^{mut/+} and BRCA1^{+/+} cells, a FACS-based cell survival assay of co-cultured cells was employed. Cells were ‘color-coded’ and tested in pairs, where one cell strain emitted a fluorescent signal (e.g. strain A, GFP+, stably transfected) and the other (strain B) did not. To insure that both cultures were cultivated in an identical environment, A and B were mixed, co-plated, and then exposed to the DNA damaging agent of choice. After 7 days of recovery, they were harvested; and the relative abundance of each cell population was analyzed by FACS (FIG. 3a). The ratio of green/non-green or non-green/green cells reflected the relative survival of the two strains.

When BRCA1^{mut/+} and BRCA1^{+/+} HMECs and fibroblast cells were compared for their sensitivity to olaparib (PI), BRCA1^{mut/+} cells (both HMECs and fibroblasts) were not found to be demonstrably sensitive (FIG. 3b, 3c). As a positive control, an HR DSBR-competent test cell line, U20S, that had been subjected to BRCA1 depletion by expression of a lentiviral BRCA1 shRNA vector (ShBRCA1), proved to be highly sensitive to olaparib, while luciferase control hairpin-transduced cells (ShLuc) were not (FIG. 3d). In addition, BRCA1^{mut/+} HMEC viability was reduced by olaparib only after BRCA1 depletion (siBRCA1, FIG. 3e). The effect of olaparib in BRCA1 shRNA-transduced U20S cells was more severe than in HMECs after BRCA1 siRNA transfection, because of the greater depletion of BRCA1 in the former, which was abetted by selecting for BRCA1-depleted cells. Nevertheless, despite their relative resistance to olaparib in the native state, BRCA1 heterozygote HMECs did become olarparib-sensitive upon further depletion of endogenous BRCA1. This result again suggests that HR function is intact in BRCA1^{mut/+} cells.

Thus, despite the linkage of HR to BRCA1 breast cancer suppression and in keeping with results obtained in mouse ES cells³⁵, these results, too, suggest that BRCA1^{mut/+} cells are not defective for HR-dependent DSBR function.

### Example 6

### Stalled Replication Fork Repair

BRCA1 also protects the genome from DNA damage resulting at stalled replication forks⁴⁰⁻⁴³. It is rapidly attracted to these damage sites where, like in HR-DSBR, it joins other proteins that are required for stalled fork damage-associated repair (SFR). For example, BRCA1 is required for the generation of phospho-RPA32-coated single stranded DNA (ssDNA), a pre-repair step needed for the recruitment to these structures of ATRIP/ATR to activate the intra-S and G2/M checkpoints that support SFR^{5,42,44-47}.

In the absence of BRCA1, a stalled fork is more likely to be bypassed by translesional synthesis⁴² (a mutagenic process), or, it may collapse into DSB, a hallmark of ‘replication stress’ (RS) and an established force in support of epithelial cancer development^{48,49}. In the mammary epithelium, which undergoes normal periods of extreme proliferation (for e.g. during pubertal development and/or pregnancy), an accumulation of stalled forks, when not resolved is likely to result in significant replication stress.

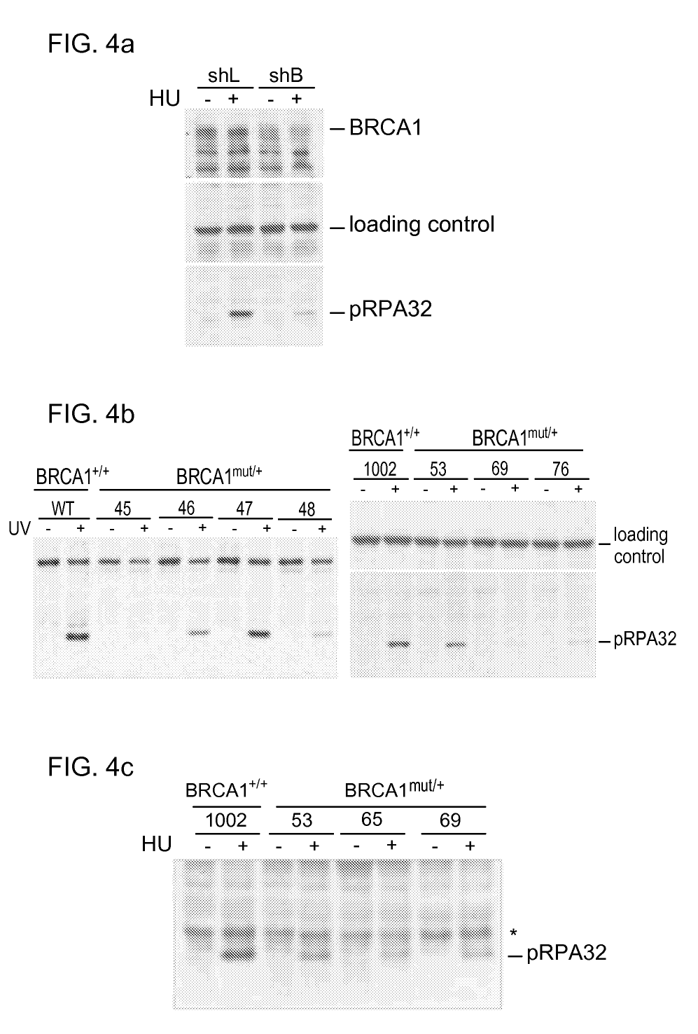

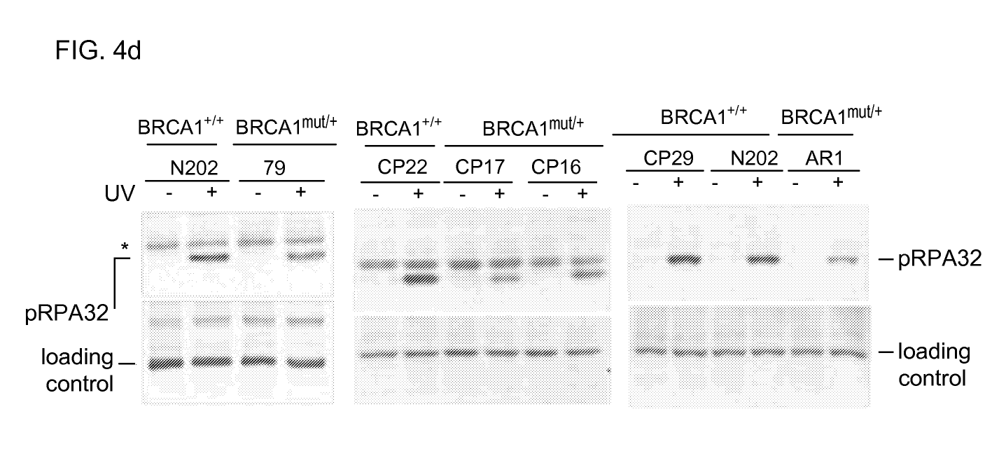

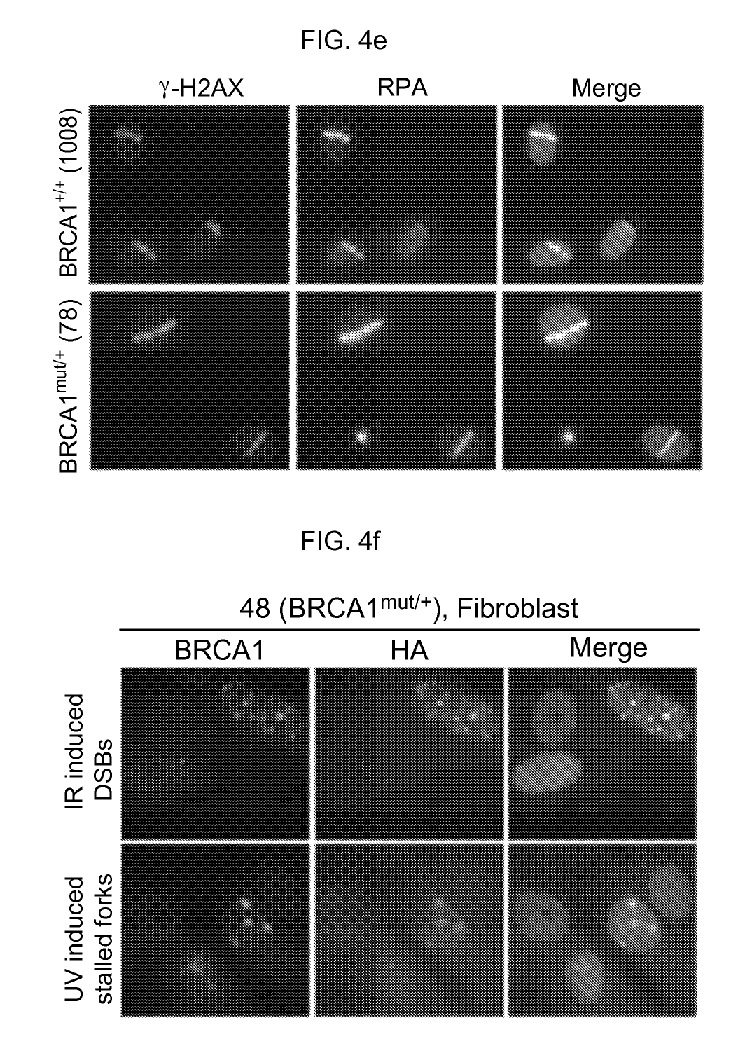

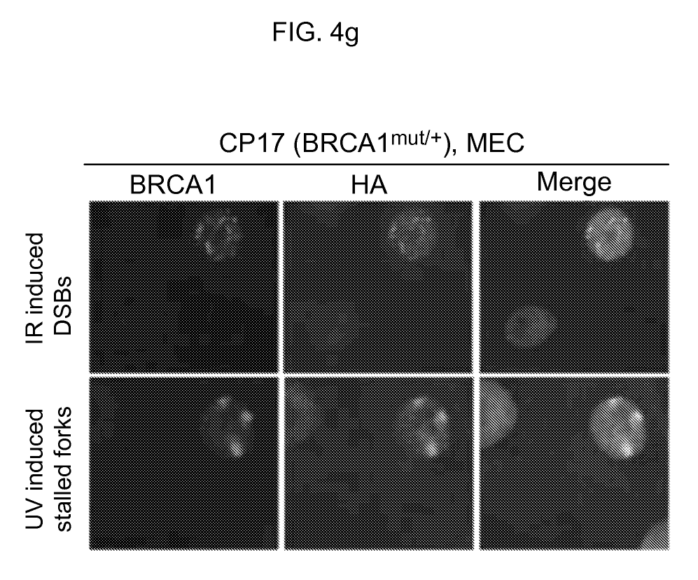

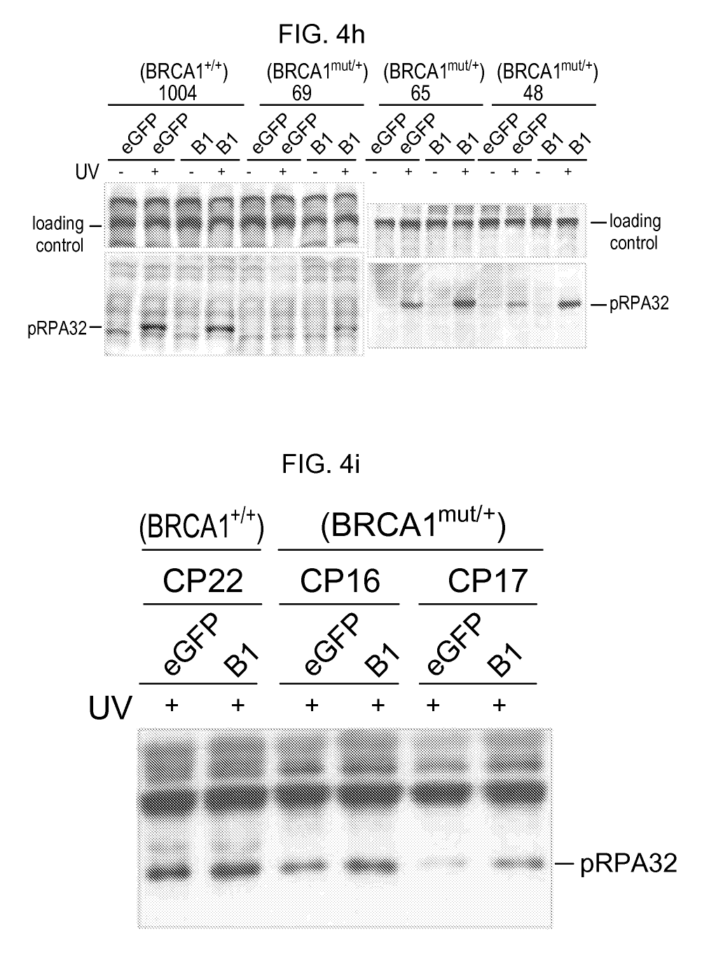

Thus, we asked whether BRCA1^{mut/+} cells are haploinsufficient in their ability to support SFR. Employing validated assays, we found that by comparison with control cells BRCA1^{mut/+} fibroblasts and HMECs were defective in their SFR responses to replication-stalling agents like HU (hydroxyurea) and UV-C (ultraviolet radiation). We have shown previously that in BRCA1^{+/+} cells that were heavily depleted of BRCA1, recruitment of phospho-RPA32 (pRPA32) to chromatin was defective in response to stalled fork inducing-agents like UV⁴². This defect was also evident after treatment with HU, another stalled fork-inducing agent (FIG. 4a). When BRCA1^{mut/+} cells were tested for their ability to recruit pRPA32 to single-stranded DNA (ssDNA) after UV and/or HU treatment, a defect, albeit incomplete, was detected in BRCA1^{mut/+} HMECs and fibroblasts (FIG. 4b, c, d,).

To rule out the possibility that inefficient loading of RPA at stalled forks in BRCA1^{mut/+} cells is a reflection of innately reduced RPA activation after DNA damage, we assayed for RPA recruitment to DNA in response to UV laser+halogenated pyrimidine-induced DSBs present in stripes. As shown in FIG. 4e, RPA was equivalently recruited to these structures in BRCA1^{mut/+} and +/+ cells. This rules out the possibility of an innate defect in RPA activation after DNA damage.

To test whether these abnormal RPA binding observations in BRCA1^{mut/+} cells are specifically linked to BRCA1 haploinsufficiency, we asked whether ectopic wt BRCA1 expression in BRCA1^{mut/+} cells corrects them. Infection by a lentiviral-BRCA1 coding vector led to wt BRCA1 expression in primary BRCA1^{mut/+} fibroblasts and HMECs (FIG. 4f, g; FIG. 10d). This product was HA-tagged (FIG. 4f, g; FIG. 10d), and, after expression, it was recruited to DSBs and stalled forks in HMECs and fibroblasts like endogenous wt BRCA1 (FIG. 4f, g). Expression of this protein in BRCA1^{mut/+} cells suppressed the defect in pRPA32 recruitment to chromatin in UV-treated fibroblasts and HMECs (FIG. 4h and i, respectively). Thus, this defect is a valid representation of BRCA1 haploinsufficiency.

An inability to form pRPA32-coated ssDNA after DNA damage may result in relevant checkpoint defects. Although we detected an incomplete reduction in pRPA32-coated chromatin after UV-induced DNA damage in BRCA1^{mut/+} HMECs, there was no obvious S or G2 checkpoint defect. Thus, incomplete formation of pRPA32-coated ssDNA, in the conditions tested, was, nonetheless, sufficient to initiate a proper checkpoint response.

Given that inefficient loading of pRPA32 on ssDNA is associated with a stalled fork repair defect, we asked whether BRCA1^{mut/+} HMEC and fibroblast strains also experience an abnormally high frequency of collapsed forks compared to their WT counterparts (BRCA1^{+/+}) after a low dose of UV (5 J/m2). Fork collapse can be captured by staining the cells with antibody to 53BP1, which is routinely recruited to these damaged structures^{42,50}.

Both BRCA1^{mut/+} HMECs and fibroblasts, stained 18 hrs post UV with monospecific p-S1778 53BP1 Ab, revealed an increase in fork collapse by comparison with wt controls (FIG. 5a, b). Nearly all BRCA1^{mut/+} strains revealed this increase (FIG. 5a, b). This again implies that the efficiency of stalled fork repair is compromised in BRCA1^{mut/+} cells, leading to higher fork collapse and incomplete resolution/repair of these structures. Thus, BRCA1 is haploinsufficient for the suppression of replication stress in primary HMECs and fibroblasts.

Exceptions were strains carrying the 185delAG mutation (study ID number: 26, 47, 53, 57 and 83). These lines exhibited near normal loading of pRPA32 onto chromatin after UV (FIG. 4b, and FIG. 10a), but more abundant 53BP1 foci by comparison with control cells tested in parallel (FIG. 5b). This suggests that a product of this allele is competent for the BRCA1 function that promotes long ssDNA development at stalled forks but incompetent for collapsed fork prevention. Others have shown that the 185delAG allele expresses a modestly truncated BRCA1 protein, translation of which is initiated immediately downstream of the mutation near the 5′ end of the gene⁵¹. Thus, one might hypothesize that 185delAG is a hypomorph, capable of supporting some but not all BRCA1 functions in support of stalled fork repair.

To further test the conclusion that inefficient stalled fork repair (SFR) in BRCA1^{mut/+} cells results in increased DNA breaks, we employed whole cell alkaline single-cell-gel electrophoresis (comet assays) to quantify the extent of DNA damage in individual cells after exposure to stalled fork-inducing agents. In the absence of UV the amount of DNA in comet tails (FIG. 5c) was insignificantly different between BRCA1^{mut/+} and BRCA1^{+/+} HMECs (FIG. 5d, left panel). However, in UV-treated cells there was a greater increase in DNA breaks in BRCA1^{mut/+} when compared to BRCA1^{+/+} cells, (FIG. 5d, right panel). These data confirm that stalled fork damage in BRCA1 heterozygous cells results in a significant increase in net DNA strand breakage. This result reaffirms the finding that, faced with replication stalling, BRCA1 heterozygous primary cells exhibit signs of replication stress.

### Example 7

### Semi-Quantitative Comparison of Cell Sensitivity To Different DNA Damage-Inducing Agents

In an effort to validate the observation that primary BRCA1^{mut/+} cells are defective for SFR and suppression of replication stress, the relative sensitivity of these primary cells to stalled fork-inducing agents, like UV and cisplatin, was tested. FACS-based quantitative assay of differentially colored, co-cultured cells was again employed. In numerous comparisons of primary BRCA1^{mut/+} and BRCA1^{+/+} fibroblasts and HMECs, the heterozygotes were significantly more sensitive to UV than the wt cells (FIG. 5e, f). Excessive sensitivity was also observed for cisplatin (FIG. 5g, h), another stalled fork inducing agent⁵²⁻⁵⁴. This evidence further reinforces the finding that primary BRCA1^{mut/+} cells are haploinsufficient for SFR.

### Example 8

### Emerging HR-DSBR Incompetence In BRCA1^{MUT/1+} Cells That Experience Excessive Replication Stress

The discordance between multiple intact and one defective BRCA1-associated functions in numerous, primary heterozygous cell strains suggests that BRCA1^{mut/+} cells exhibit a hierarchy among these BRCA1-dependent functions. The data imply that heterozygous mutant cells preferentially direct their limited stores of intact BRCA1 protein to checkpoint activation, HR-DSBR, centrosome, and spindle pole function and less effectively to stalled fork repair (SFR). Alternatively, less BRCA1 protein is required for the former than the latter function. In either case, we asked whether, when these cells encounter sufficient replication stress, BRCA1 becomes preferentially dedicated to SFR and, in doing so, reduces the pool of uncommitted BRCA1 available for an otherwise intact function, like HR-DSBR. If it falls sufficiently, do BRCA1^{mut/+} cells now become multiply haploinsufficient—i.e. for SFR, HR-DSBR, and possibly other known BRCA1 functions that were formerly intact in these cells.

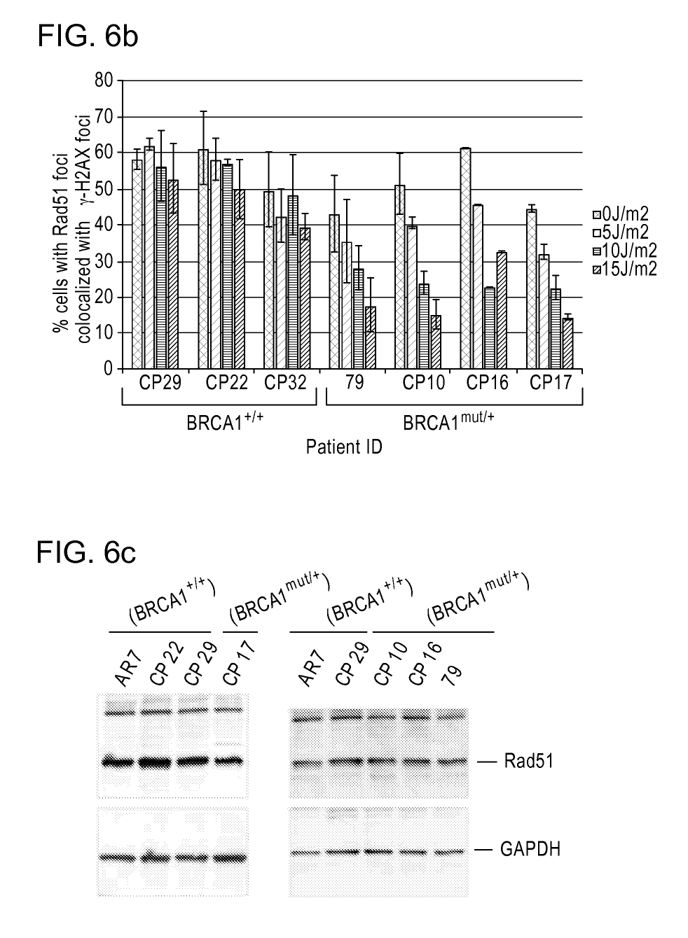

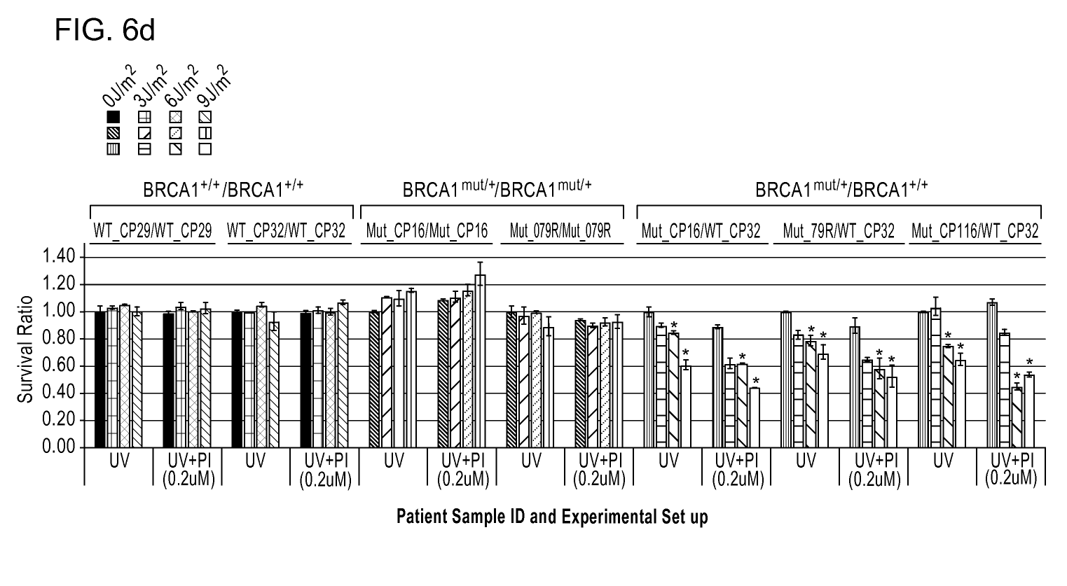

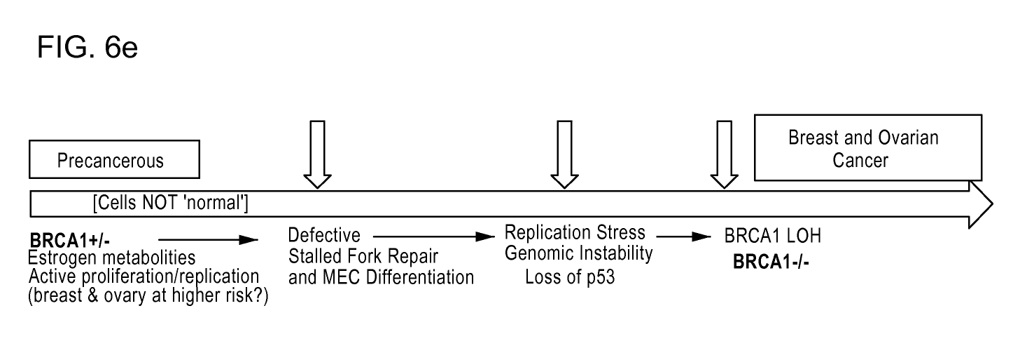

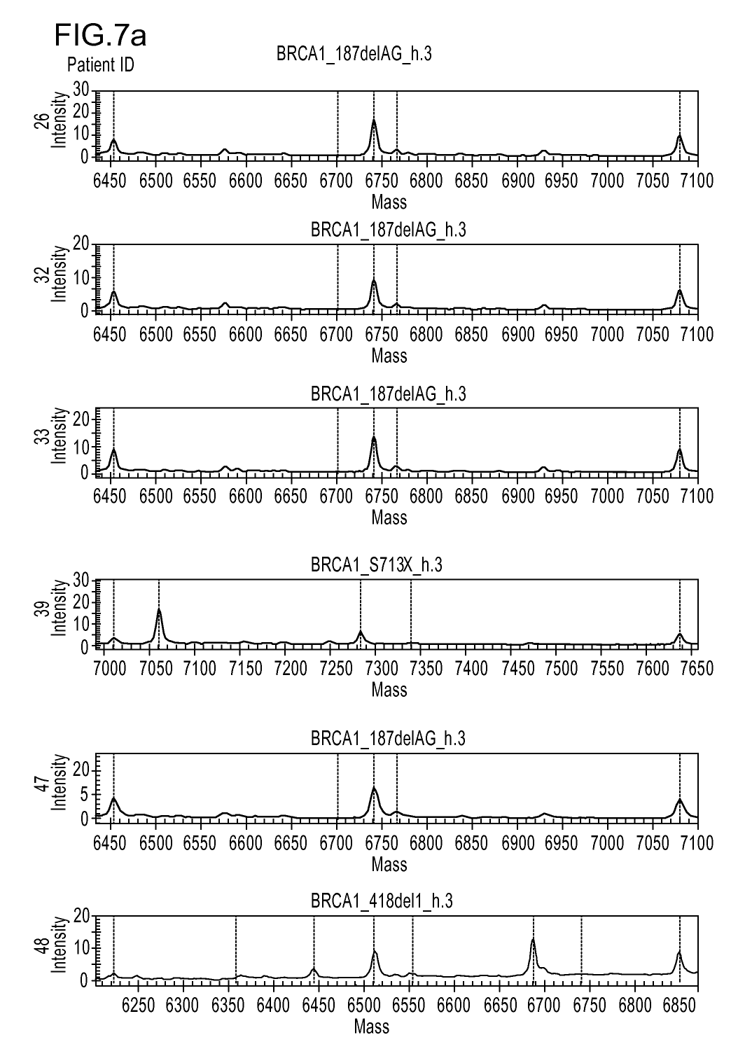

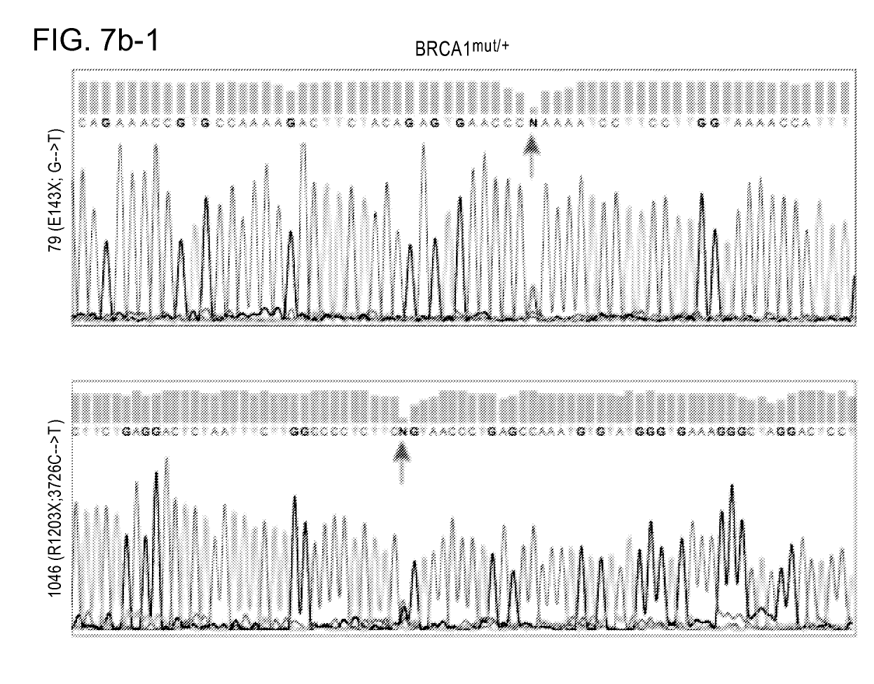

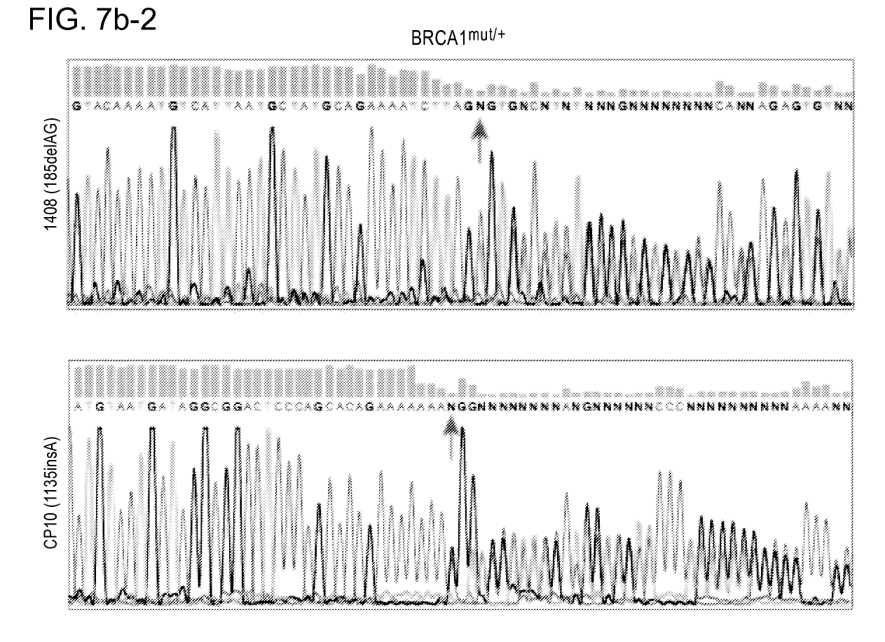

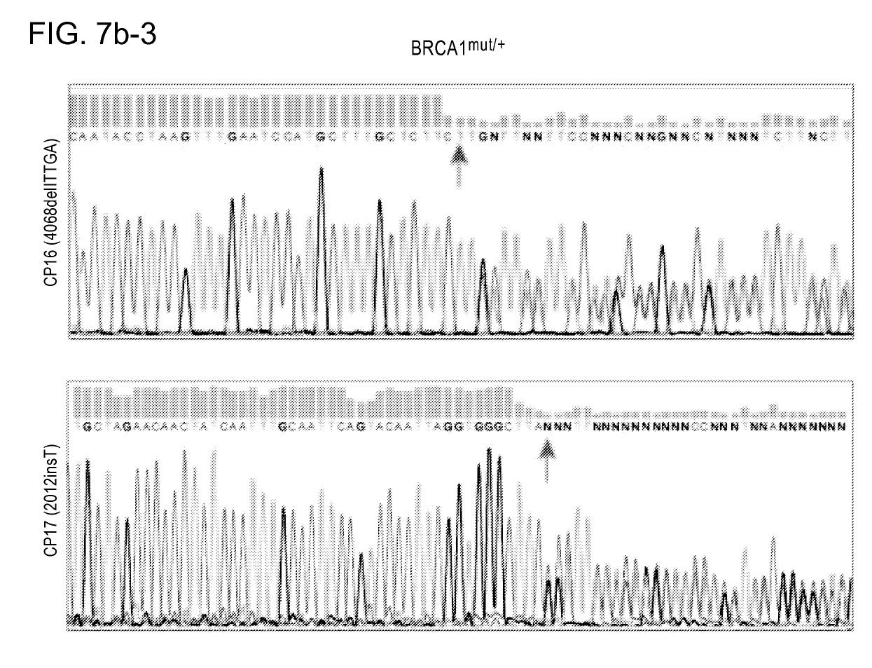

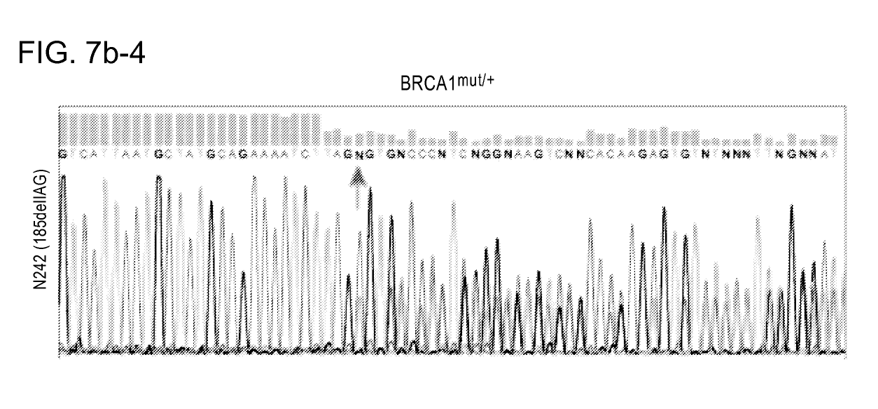

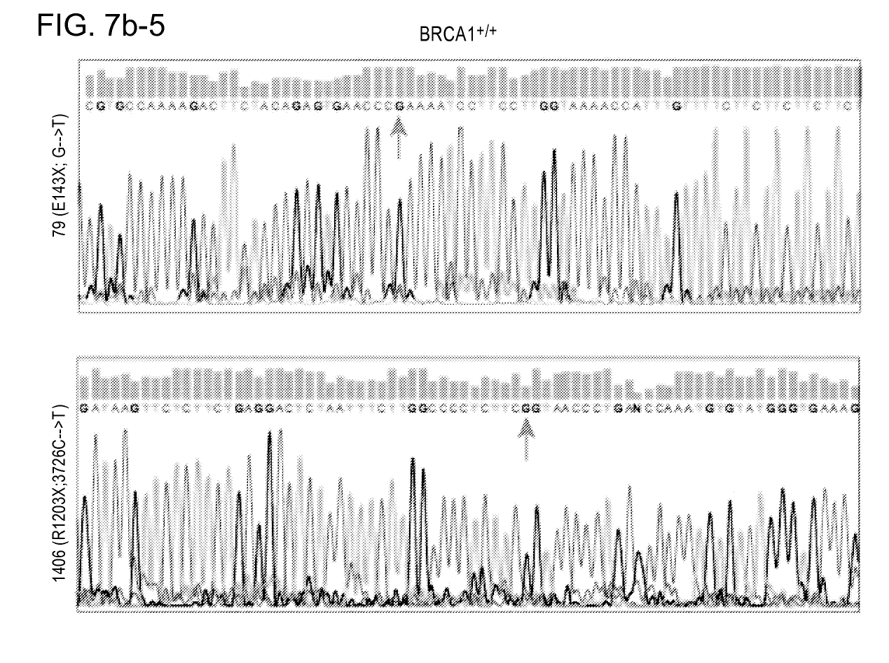

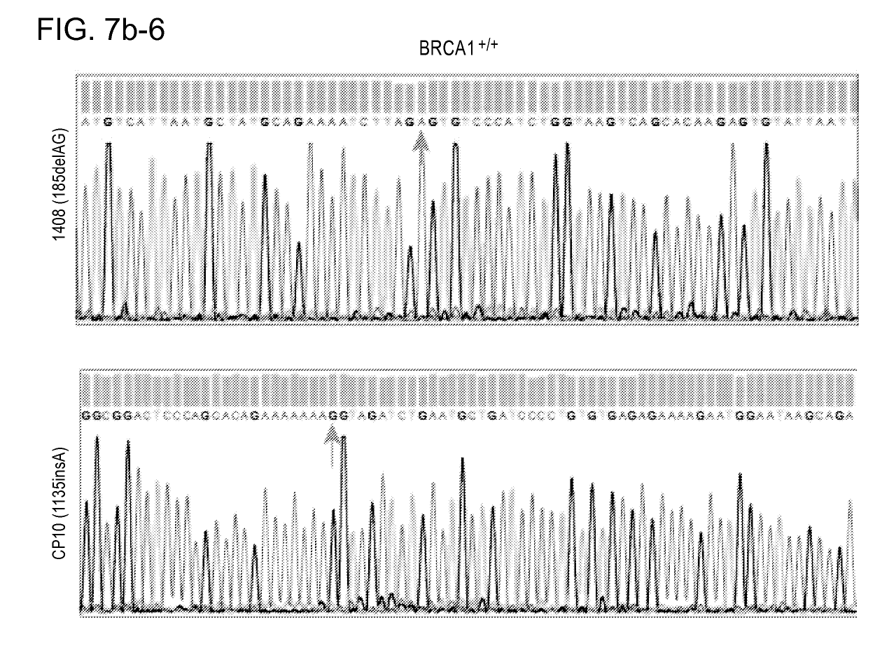

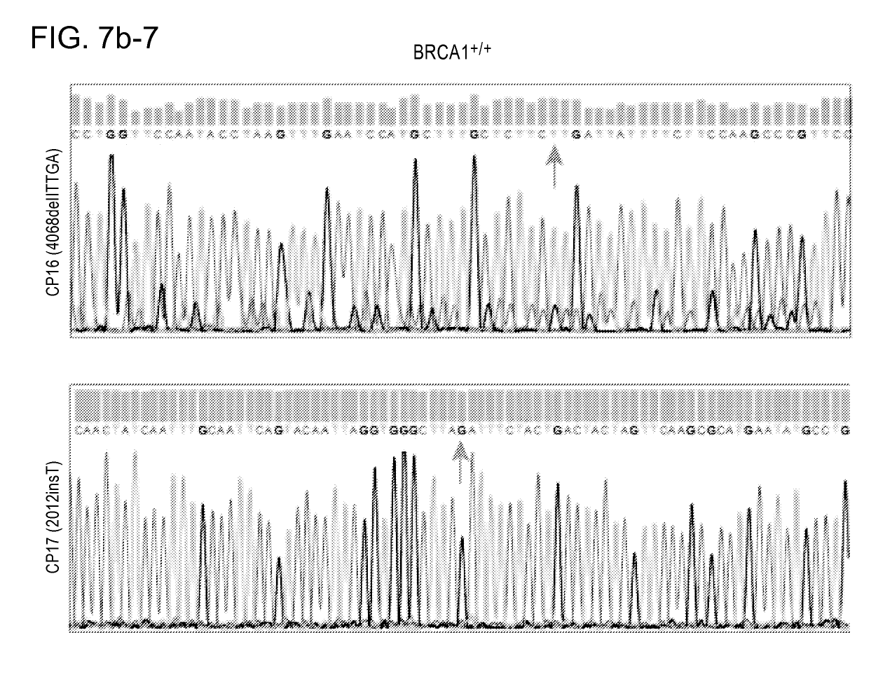

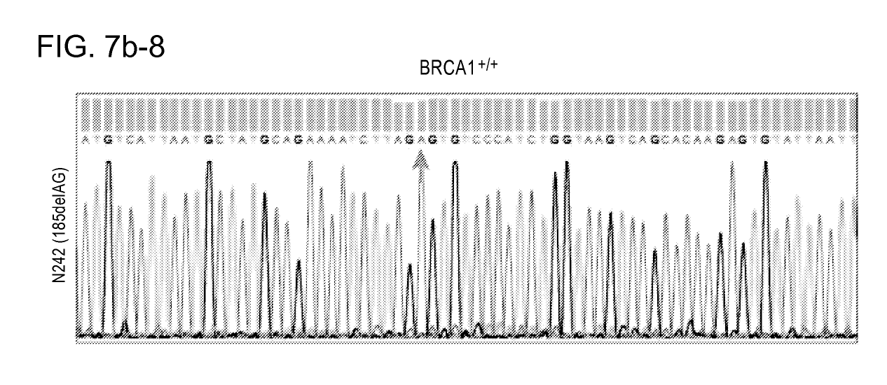

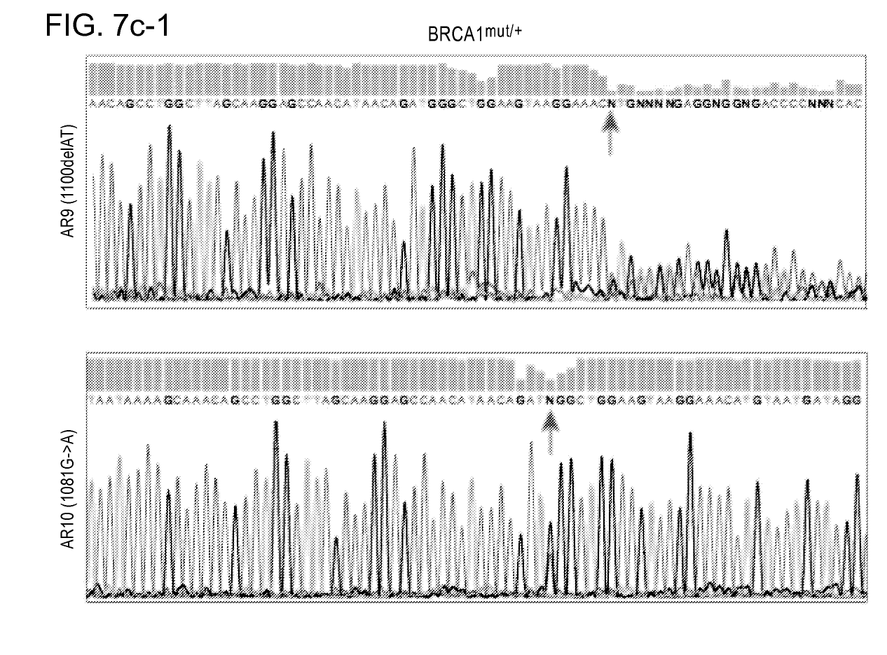

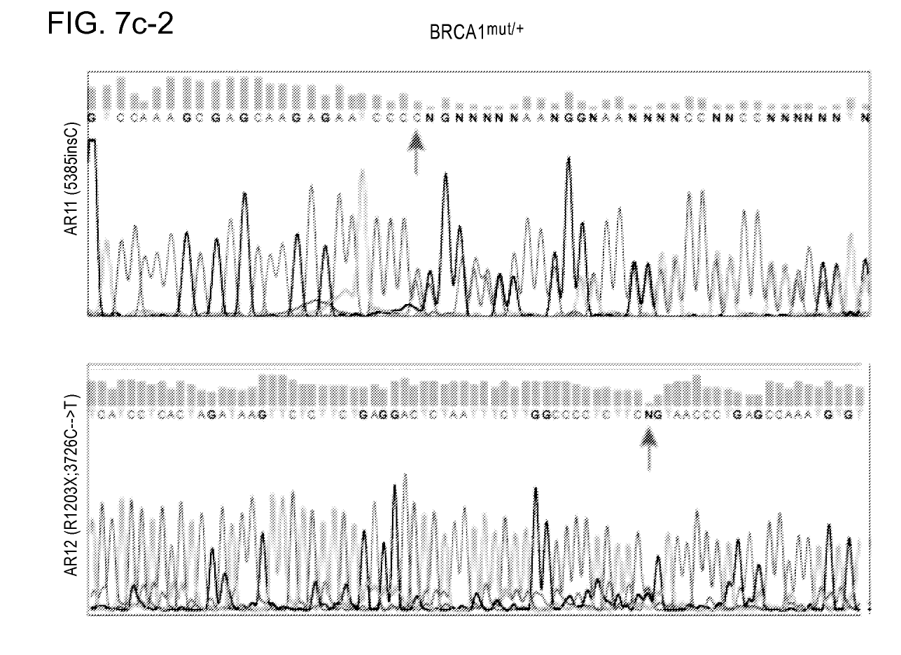

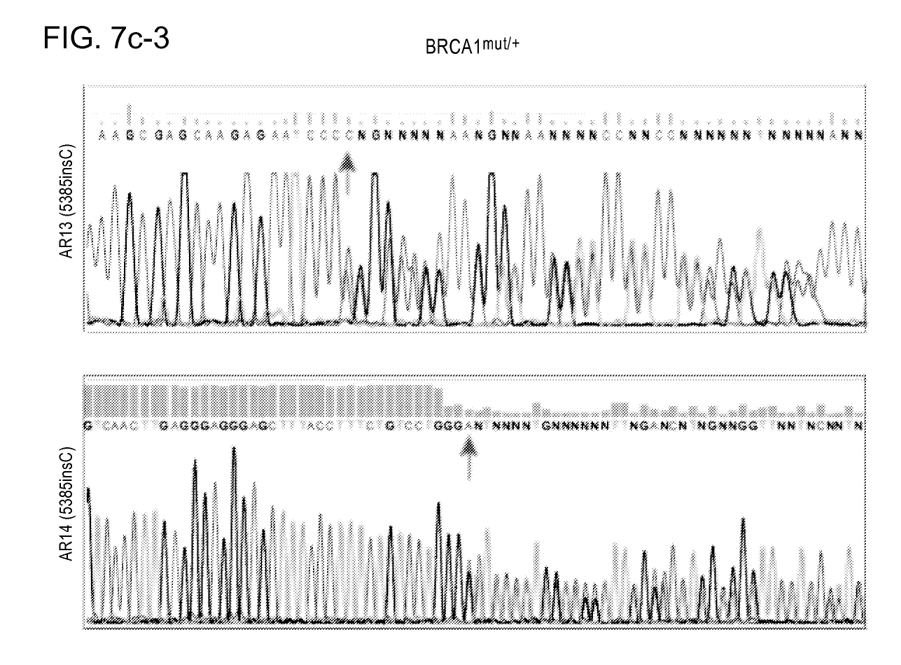

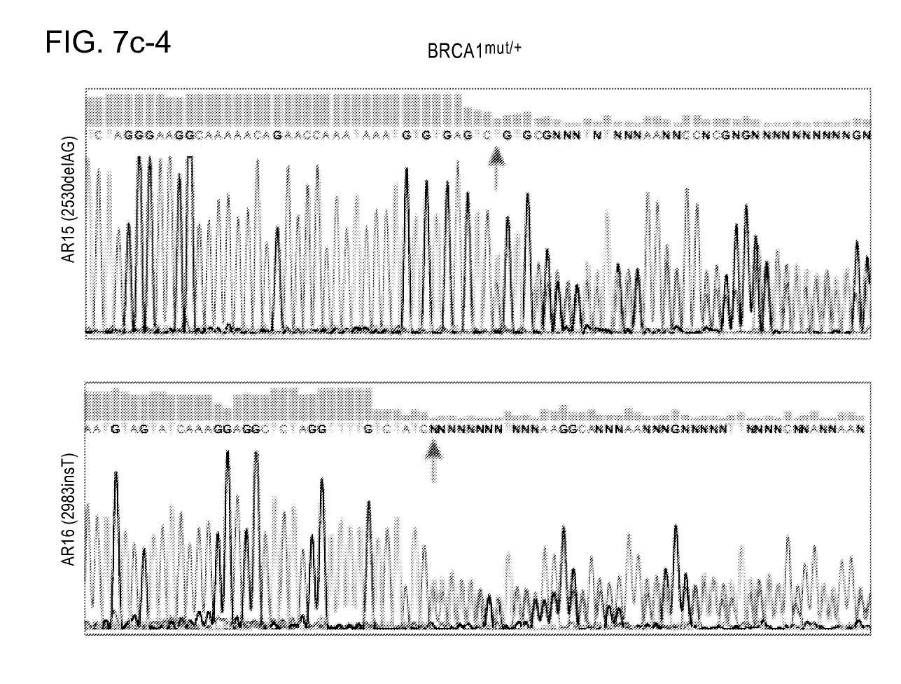

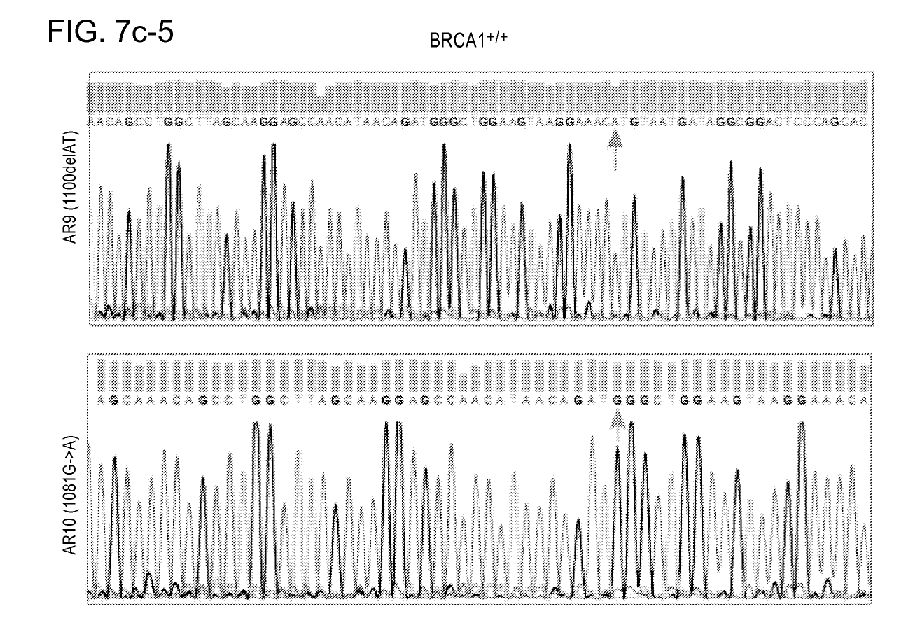

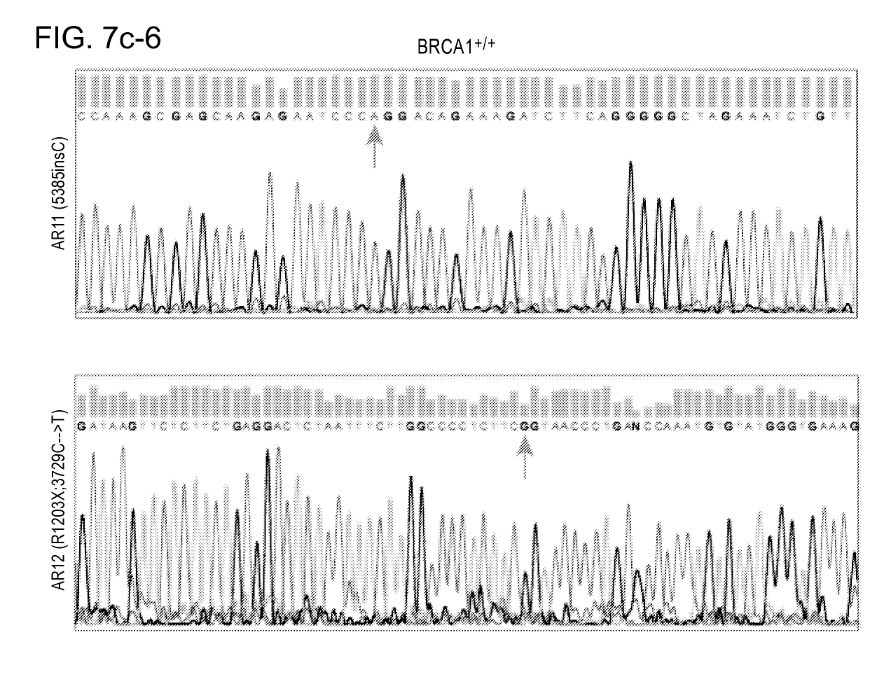

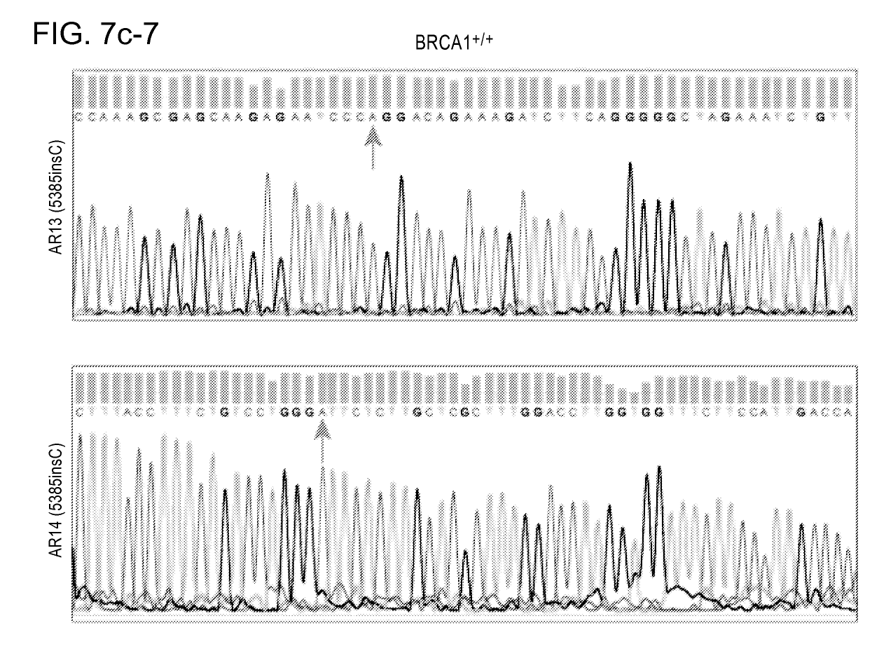

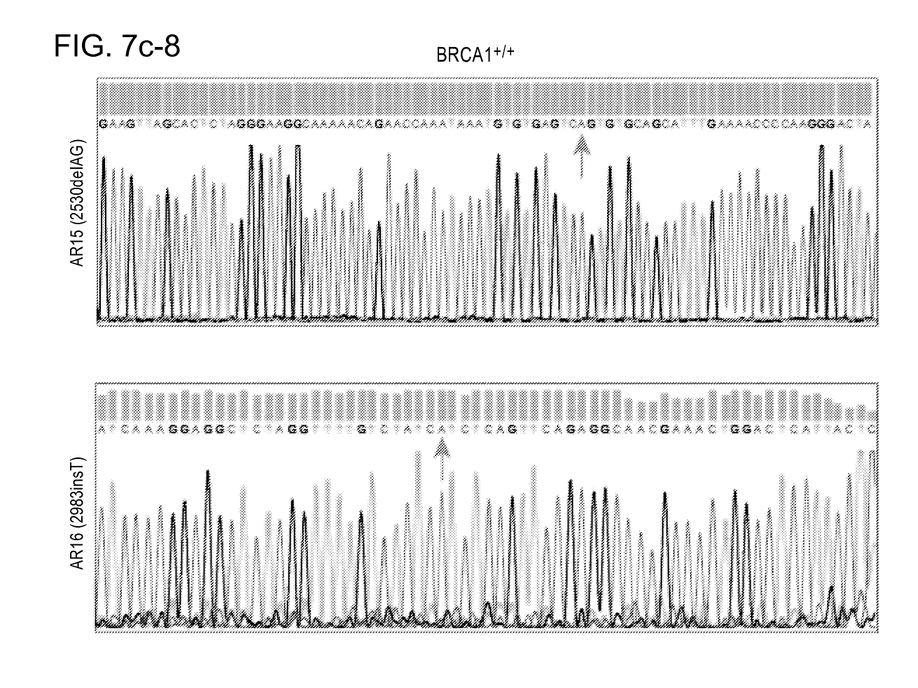

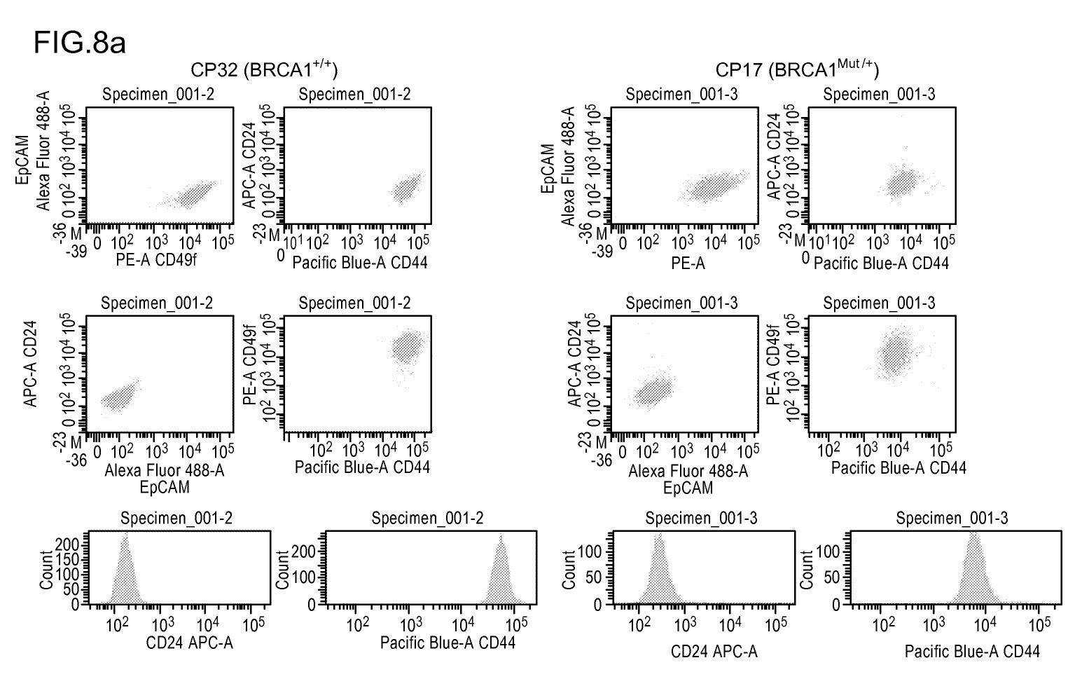

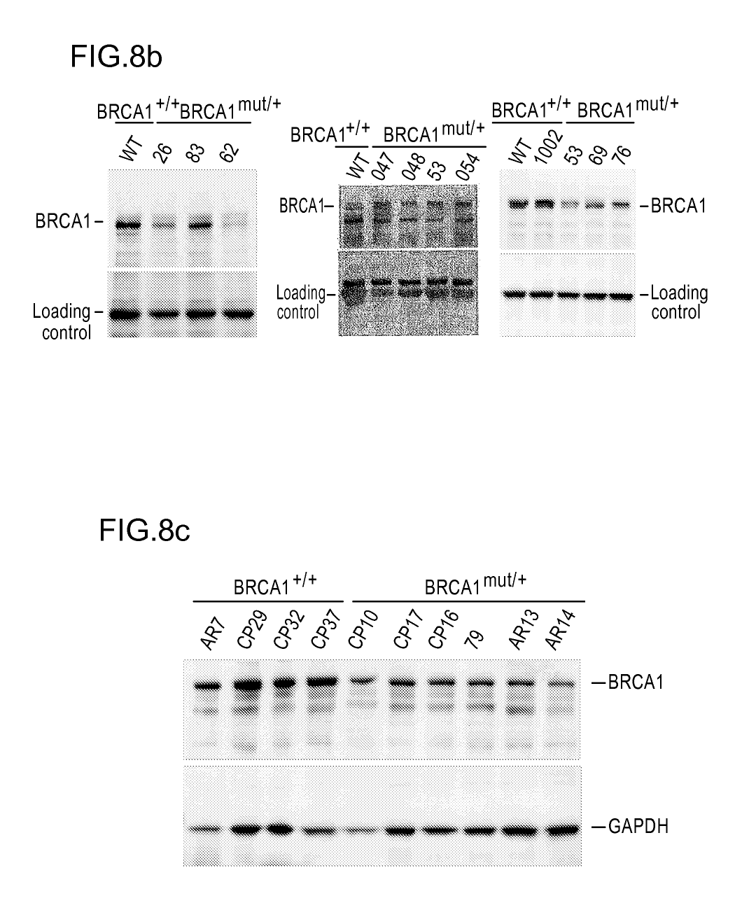

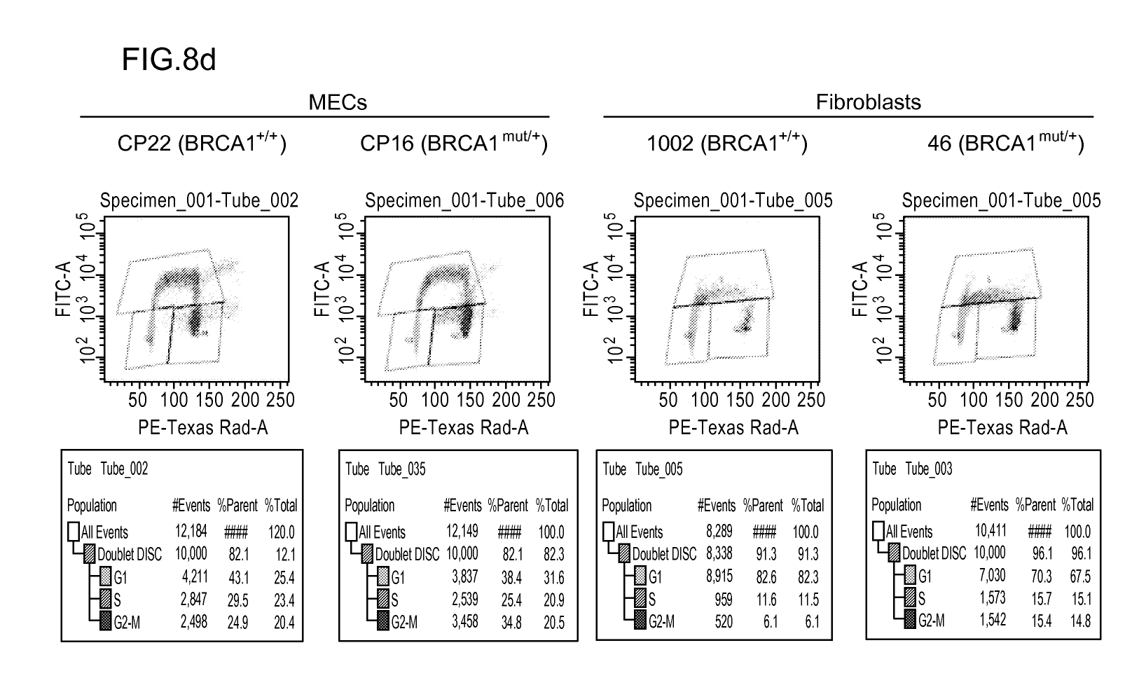

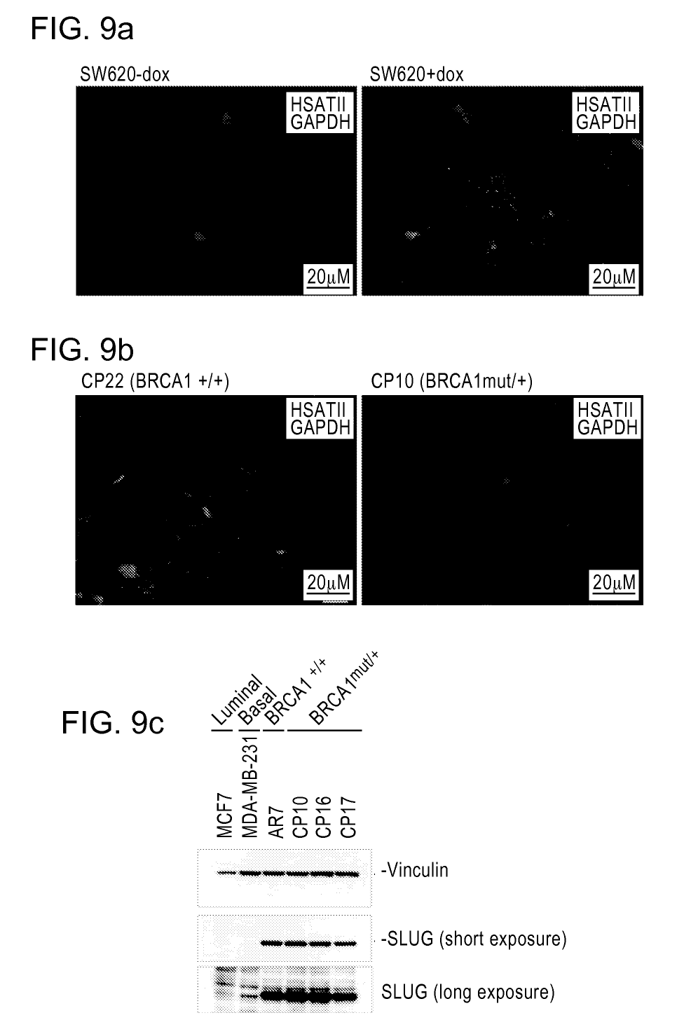

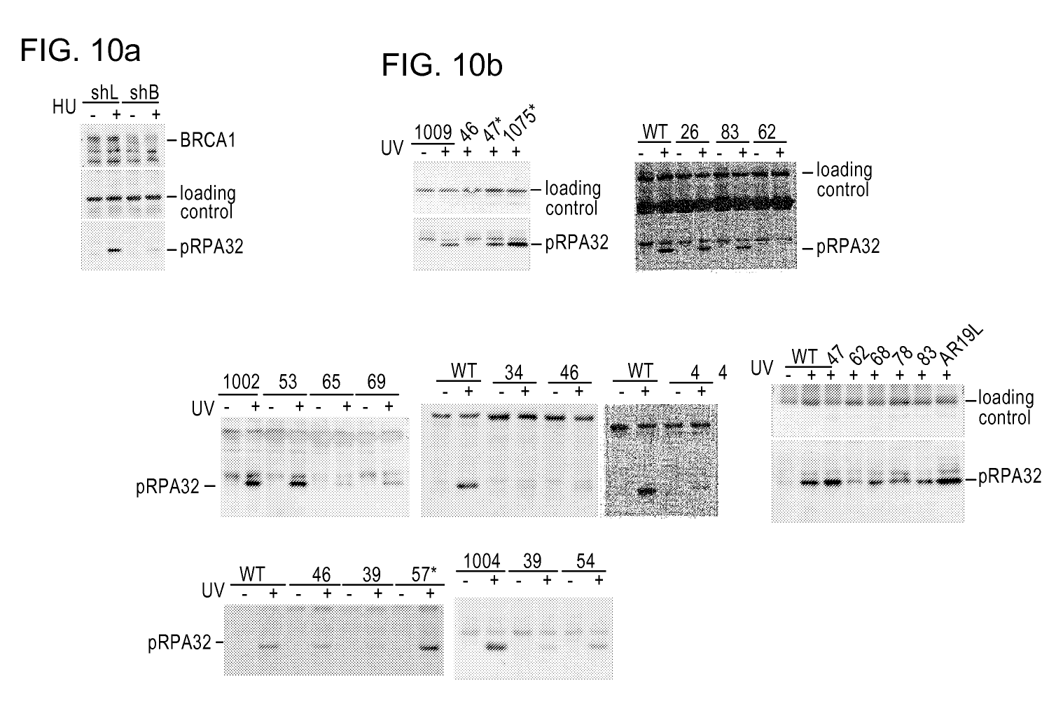

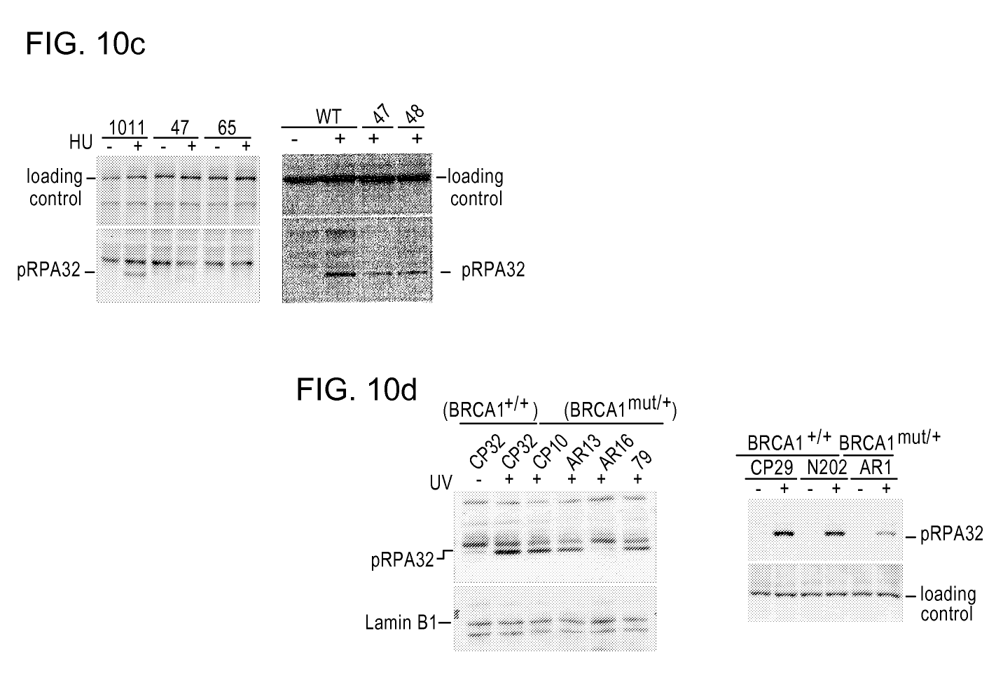

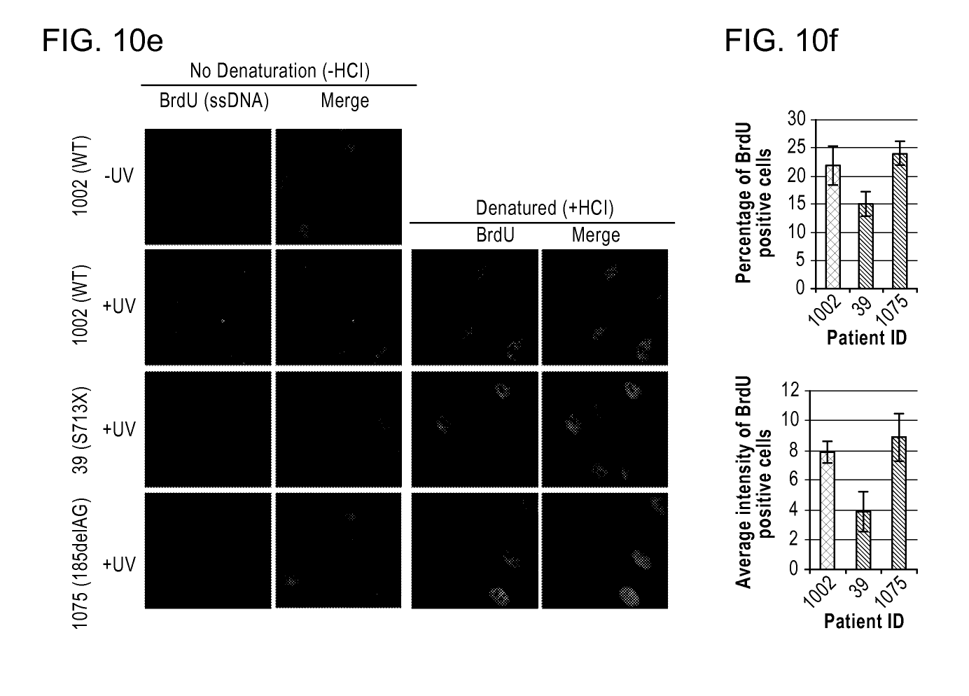

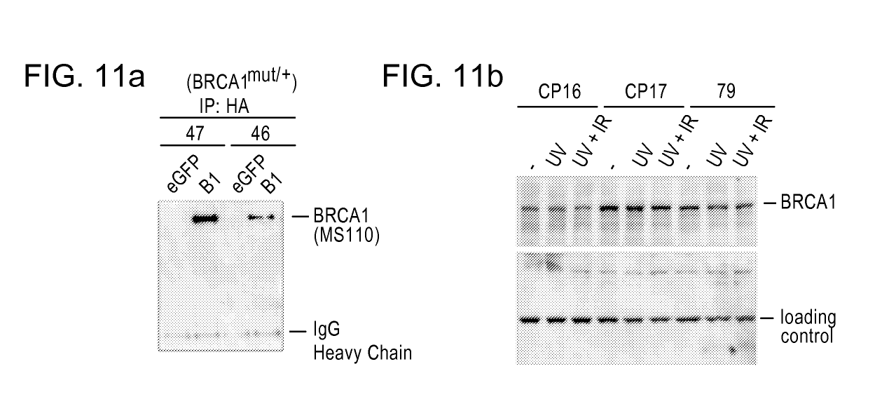

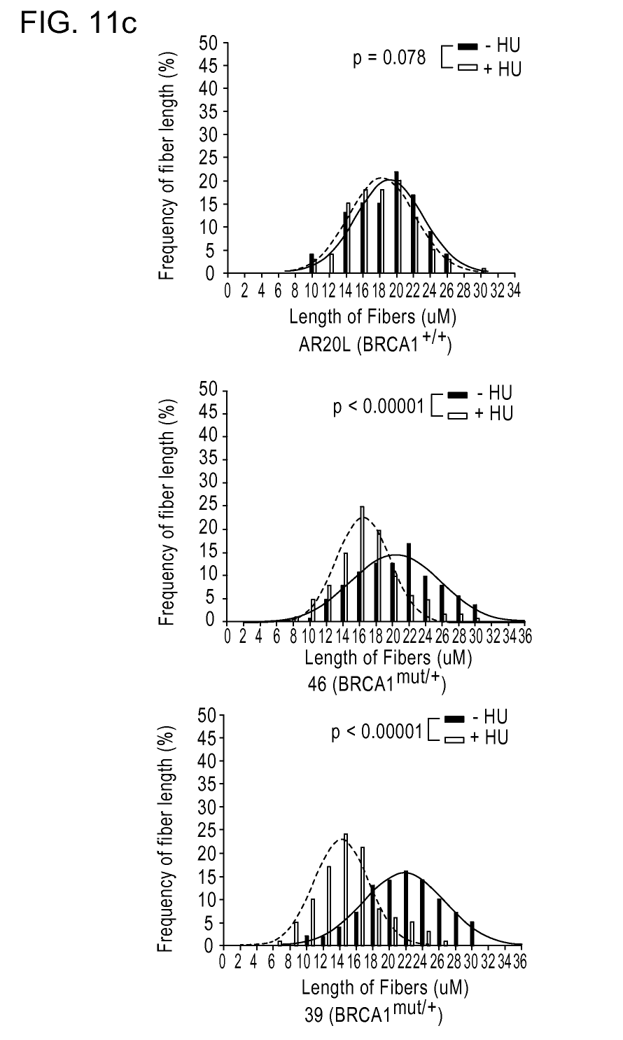

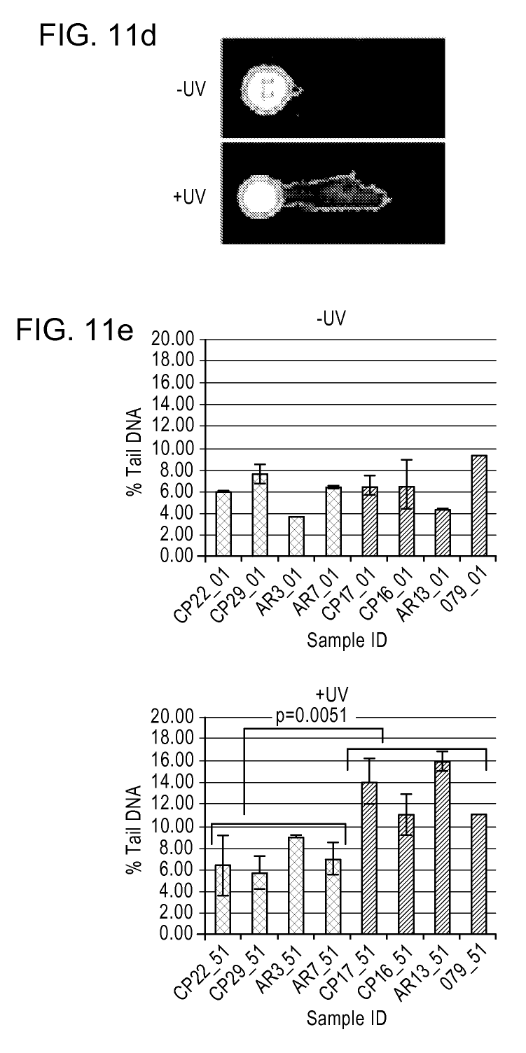

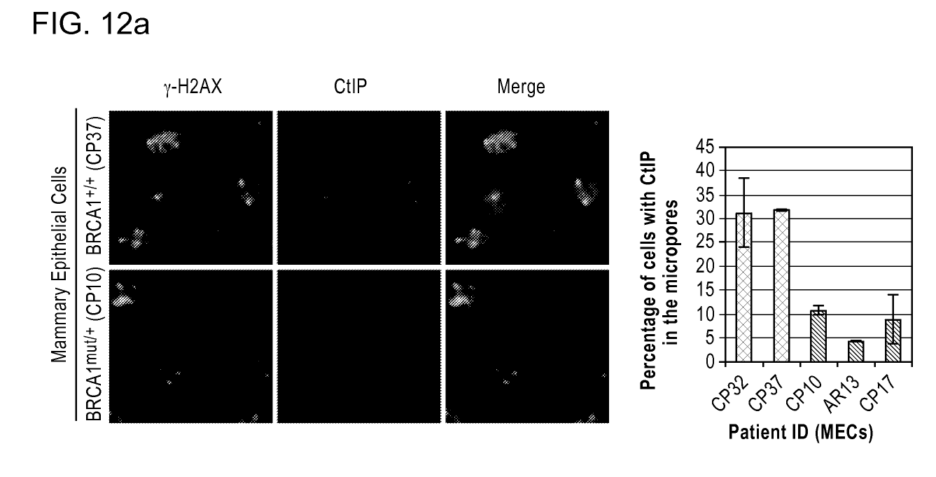

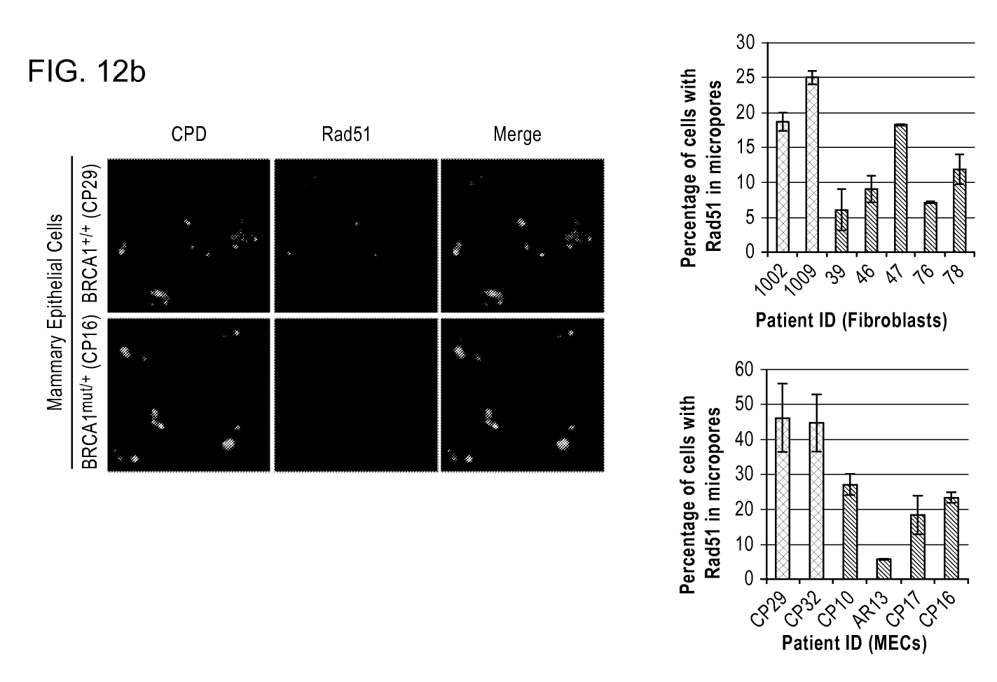

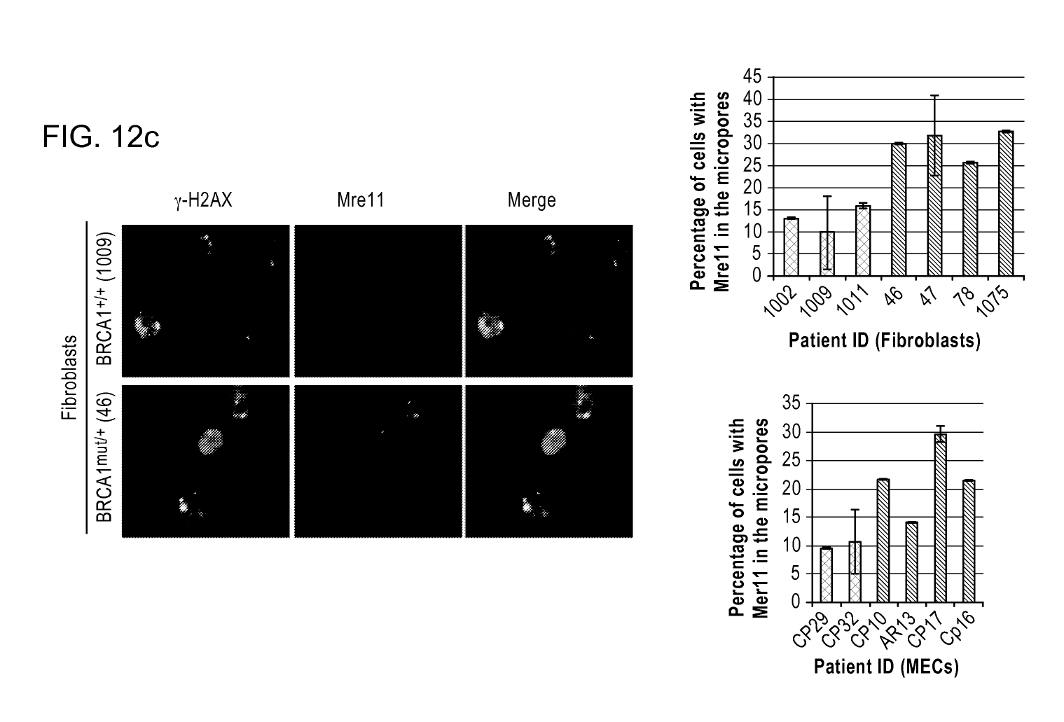

To address these questions, we pre-exposed cells (both BRCA1^{mut/+} and BRCA1^{+/+}) to increasing doses of UV (5, 10, 15 J/m2) and then assayed them for other BRCA1 functions (other than SFR). To assay for HR, the UV-treated cells were allowed to recover for 1 hr and then irradiated with IR (10Gy) and analyzed for recruitment of Rad51 to DSBs (FIG. 6a). To assay for spindle formation efficiency and centrosome maintenance we allowed the cells to recover for one and/or two full cycles of cell division (24 and/or 48 hours post UV, respectively) and then analyzed the cells for spindle poles and spindles as well as centrosomes.

As shown above in FIGS. 2e, f, multiple BRCA1^{mut/+} and BRCA1^{+/+} cell strains recruited Rad51 to IR-induced DSBs with equal efficiency prior to UV pre-treatment. However in UV pre-treated cells, the ability of BRCAr^{mut/+} cells, to recruit Rad51 to DSBs became increasingly defective after exposure to increasing doses of UV (5 J, 10 J, 15 J; FIG. 6a and b). No such effect was detected in BRCA1^{+/+} cells. Given that BRCA1 is required for recruitment of Rad51 to sites of DSBs, we asked whether changes in BRCA1 protein levels in BRCA1 heterozygotes after treatment with UV could account for reduced Rad51 recruitment in UV-pretreated BRCA1^{mut/+} HMECs. No obvious alterations in BRCA1 protein levels were observed in BRCA1 heterozygotes after UV treatment (FIG. 10e). This result, along with the observation that Rad51 protein levels in BRCA1^{mut/+} and BRCA1^{+/+} were also similar (FIG. 6c), suggests that defect in Rad51 recruitment to BRCA1-containing foci, i.e. DSB, in UV-pretreated BRCA1^{mut/+} cells is a result of a breakdown in the ability of a limited pool of BRCA1 protein to respond to DSBs by HR-DSBR.

To assess further the apparent emergence of ‘conditional haploinsufficiency’ for HR-DSBR in the presence of replication stress, we used the FACS-based assay described earlier to determine the survival efficiency of BRCA1^{mut/+} cells in presence and absence of the Parp inhibitor, olaparib (PI). As shown in FIG. 3b, c, BRCA1^{mut/+} cells were not overtly sensitive to olaparib; so the question here was whether pre-exposure of cells to stalled fork-inducing damage (e.g. after UV exposure) compromised the ability of these cells to carry out DSBR. If so, the BRCA1^{mut/+} cells should become olaparib-sensitive. Evidence presented in FIG. 6d showed this to be the case. Exposure of BRCA1^{mut/+} cells to increasing doses of UV before adding olaparib (PI) rendered them acutely sensitive to a relatively low concentration of the PI (FIG. 6d).

Centrosome number and spindle formation in the same cell strains were not altered under these conditions (data not shown). This implies that, at the very least, there is conditional haploinsufficiency⁵⁵ for HR-DSBR and not for the other BRCA1 functions that were tested in BRCA1^{mut/+} cells facing sufficient replication stress.

### REFERENCES

1. Roy, R., Chun, J. & Powell, S. N. BRCA1 and BRCA2: different roles in a common pathway of genome protection. Nat Rev Cancer 12, 68-78 (2011).
2. Venkitaraman, A. R. Cancer susceptibility and the functions of BRCA1 and BRCA2. Cell 108, 171-182 (2002).
3. Harper, J. W. & Elledge, S. J. The DNA damage response: ten years after. Mol Cell 28, 739-745 (2007).
4. Osborn, A. J., Elledge, S. J. & Zou, L. Checking on the fork: the DNA-replication stress-response pathway. Trends Cell Biol 12, 509-516 (2002).
5. Zeman, M. K. & Cimprich, K. A. Causes and consequences of replication stress. Nature cell biology 16, 2-9 (2013).
6. Walsh, T. & King, M.-C. Ten genes for inherited breast cancer. in Cancer Cell, Vol. 11 103-105 (2007).
7. Scully, R. & Livingston, D. M. In search of the tumour-suppressor functions of BRCA1 and BRCA2. Nature 408, 429-432 (2000).
8. King, M.-C., Marks, J. H., Mandell, J. B. & Group, N.Y.B.C.S. Breast and ovarian cancer risks due to inherited mutations in BRCA1 and BRCA2. Science 302, 643-646 (2003).
9. Narod, S. A. & Foulkes, W. D. BRCA1 and BRCA2: 1994 and beyond. Nat Rev Cancer 4, 665-676 (2004).
10. Walsh, T. & King, M.-C. Ten genes for inherited breast cancer. Cancer Cell 11, 103-105 (2007).
11. Greenberg, R. A., et al. Multifactorial contributions to an acute DNA damage response by BRCA1/BARD1-containing complexes. Genes Dev 20, 34-46 (2006).
12. Huen, M. S. Y., Sy, S. M. H. & Chen, J. BRCA1 and its toolbox for the maintenance of genome integrity. Nat Rev Mol Cell Biol (2009).
13. Wang, B., et al. Abraxas and RAP80 form a BRCA1 protein complex required for the DNA damage response. Science 316, 1194-1198 (2007).
14. Silver, D. P. & Livingston, D. M. Mechanisms of BRCA1 Tumor Suppression. Cancer Discovery (2012).
15. Martins, F. C., et al. Evolutionary Pathways in BRCA1-Associated Breast Tumors. Cancer Discovery (2012).
16. Lim, E., et al. Aberrant luminal progenitors as the candidate target population for basal tumor development in BRCA1 mutation carriers. Nat Med 15, 907-913 (2009).
17. Liu, S., et al. BRCA1 regulates human mammary stem/progenitor cell fate. Proc Natl Acad Sci USA 105, 1680-1685 (2008).
18. Molyneux, G., et al. BRCA1 basal-like breast cancers originate from luminal epithelial progenitors and not from basal stem cells. Cell stem cell 7, 403-417 (2010).
19. Proia, T. A., et al. Genetic predisposition directs breast cancer phenotype by dictating progenitor cell fate. Cell stem cell 8, 149-163 (2011).
20. Burga, L. N., et al. Altered proliferation and differentiation properties of primary mammary epithelial cells from BRCA1 mutation carriers. Cancer Res 69, 1273-1278 (2009).
21. Choudhury, S., et al. Molecular profiling of human mammary gland links breast cancer risk to a p27(+) cell population with progenitor characteristics. Cell stem cell 13, 117-130 (2013).
22. MacConaill, L., et al. Profiling critical cancer gene mutations in clinical tumor samples. PLOS ONE 4, e7887 (2009).
23. Thomas, R. K., et al. High-throughput oncogene mutation profiling in human cancer. Nature genetics 39, 347-351 (2007).
24. Keller, P. J., et al. Mapping the cellular and molecular heterogeneity of normal and malignant breast tissues and cultured cell lines. Breast cancer research: BCR 12, R87 (2010).
25. Kais, Z., et al. KIAA0101 interacts with BRCA1 and regulates centrosome number. Molecular cancer research: MCR 9, 1091-1099 (2011).
26. Joukov, V., et al. The BRCA1/BARD1 heterodimer modulates ran-dependent mitotic spindle assembly. Cell 127, 539-552 (2006).
27. Parvin, J. D. The BRCA1-dependent ubiquitin ligase, gamma-tubulin, and centrosomes. Environmental and molecular mutagenesis 50, 649-653 (2009).
28. Pujana, M. A., et al. Network modeling links breast cancer susceptibility and centrosome dysfunction. Nature genetics 39, 1338-1349 (2007).
29. Zhu, Q., et al. BRCA1 tumour suppression occurs via heterochromatin-mediated silencing. Nature 477, 179-184 (2011).
30. Xu, B., St, K. & Kastan, M. B. Involvement of Brca1 in S-phase and G(2)-phase checkpoints after ionizing irradiation. Mol Cell Biol 21, 3445-3450 (2001).
31. Kastan, M. B. & Bartek, J. Cell-cycle checkpoints and cancer. Nature 432, 316-323 (2004).
32. Zhong, Q., et al. Association of BRCA1 with the hRad50-hMre11-p95 complex and the DNA damage response. Science 285, 747-750 (1999).
33. Moynahan, M. E., Chiu, J. W., Koller, B. H. & Jasin, M. Brca1 controls homology-directed DNA repair. Mol Cell 4, 511-518 (1999).
34. Zhang, J. & Powell, S. N. The role of the BRCA1 tumor suppressor in DNA double-strand break repair. Mol Cancer Res 3, 531-539 (2005).
35. Moynahan, M. E., Cui, T. Y. & Jasin, M. Homology-directed dna repair, mitomycin-c resistance, and chromosome stability is restored with correction of a Brca1 mutation. Cancer Res 61, 4842-4850 (2001).
36. Konishi, H., et al. Mutation of a single allele of the cancer susceptibility gene BRCA1 leads to genomic instability in human breast epithelial cells. in Proc Natl Acad Sci USA (2011).
37. Rottenberg, S., et al. High sensitivity of BRCA1-deficient mammary tumors to the PARP inhibitor AZD2281 alone and in combination with platinum drugs. Proceedings of the National Academy of Sciences of the United States of America 105, 17079-17084 (2008).
38. Helleday, T., Bryant, H. E. & Schultz, N. Poly(ADP-ribose) polymerase (PARP-1) in homologous recombination and as a target for cancer therapy. Cell cycle (Georgetown, Tex.) 4, 1176-1178 (2005).
39. Farmer, H., et al. Targeting the DNA repair defect in BRCA mutant cells as a therapeutic strategy. Nature 434, 917-921 (2005).
40. Fang, J., Chen, T., Chadwick, B. P., Li, E. & Zhang, Y. Ring1b-mediated H2A ubiquitination associates with inactive X chromosomes and is involved in initiation of X inactivation. J Biol Chem 279, 52812-52815 (2004).
41. Li, L. BRCA1 Forks Over New Roles in DNA-Damage Response—Before and Beyond the Breaks. Mol Cell 44, 174-176 (2011).
42. Pathania, S., et al. BRCA1 Is Required for Postreplication Repair after UV-Induced DNA Damage. Mol Cell (2011).
43. Schlacher, K., Wu, H. & Jasin, M. A Distinct Replication Fork Protection Pathway Connects Fanconi Anemia Tumor Suppressors to RAD51-BRCA1/2. in Cancer Cell, Vol. 22 106-116 (2012).
44. Zou, L. & Elledge, S. J. Sensing DNA damage through ATRIP recognition of RPA-ssDNA complexes. Science 300, 1542-1548 (2003).
45. Toledo, L. I., et al. ATR Prohibits Replication Catastrophe by Preventing Global Exhaustion of RPA. Cell 155, 1088-1103 (2013).
46. Couch, F. B., et al. ATR phosphorylates SMARCAL1 to prevent replication fork collapse. Genes and Development 27, 1610-1623 (2013).
47. Cimprich, K. A. & Cortez, D. ATR: an essential regulator of genome integrity. Nat Rev Mol Cell Biol 9, 616-627 (2008).
48. Bartkova, J., et al. DNA damage response as a candidate anti-cancer barrier in early human tumorigenesis. Nature 434, 864-870 (2005).
49. Gorgoulis, V. G., et al. Activation of the DNA damage checkpoint and genomic instability in human precancerous lesions. Nature 434, 907-913 (2005).
50. Harding, S. M. & Bristow, R. G. Discordance between phosphorylation and recruitment of 53BP1 in response to DNA double-strand breaks. Cell Cycle 11, 1432-1444 (2012).
51. Buisson, M., Anczuków, O., Zetoune, A. B., Ware, M. D. & Mazoyer, S. The 185delAG mutation (c.68_69delAG) in the BRCA1 gene triggers translation reinitiation at a downstream AUG codon. Human mutation 27, 1024-1029 (2006).
52. Dronkert, M. L. & Kanaar, R. Repair of DNA interstrand cross-links. Mutat Res 486, 217-247 (2001).
53. Ghosal, G. & Chen, J. DNA damage tolerance: a double-edged sword guarding the genome. Translational cancer research 2, 107-129 (2013).
54. Sokol, A. M., Cruet-Hennequart, S., Pasero, P. & Carty, M. P. DNA polymerase η modulates replication fork progression and DNA damage responses in platinum-treated human cells. Scientific Reports 3, 3277 (2013).
55. Bartek, J., Lukas, J. & Bartkova, J. DNA damage response as an anti-cancer barrier: damage threshold and the concept of &apos; conditional haploinsufficiency &apos. Cell cycle (Georgetown, Tex.) 6, 2344-2347 (2007).
56. Lambert, S. A. E., Watson, A., Sheedy, D. M., Martin, B. & Carr, A. M. Gross chromosomal rearrangements and elevated recombination at an inducible site-specific replication fork barrier. Cell 121, 689-702 (2005).
57. Michel, B., Grompone, G., Flores, M.-J. & Bidnenko, V. Multiple pathways process stalled replication forks. Proc Natl Acad Sci USA 101, 12783-12788 (2004).
58. Saleh-Gohari, N., et al. Spontaneous homologous recombination is induced by collapsed replication forks that are caused by endogenous DNA single-strand breaks. Molecular and cellular biology 25, 7158-7169 (2005).
59. Willis, N. A., et al. BRCA1 controls homologous recombination at Tus/Ter-stalled mammalian replication forks. Nature In press(2014).
60. Savage, K. I., et al. BRCA1 deficiency exacerbates estrogen induced DNA damage and genomic instability. Cancer research (2014).
61. Lu, F., Zahid, M., Saeed, M., Cavalieri, E. L. & Rogan, E. G. Estrogen metabolism and formation of estrogen-DNA adducts in estradiol-treated MCF-10F cells. The effects of 2,3,7,8-tetrachlorodibenzo-p-dioxin induction and catechol-O-methyltransferase inhibition. The Journal of steroid biochemistry and molecular biology 105, 150-158 (2007).
62. Suzuki, N., et al. Translesion Synthesis Past Estrogen-Derived DNA Adducts by Human DNA Polymerases η and κ†. Biochemistry 43, 6304-6311 (2004).
63. Yager, J. D. & Davidson, N. E. Estrogen carcinogenesis in breast cancer. The New England Journal of Medicine 354, 270-282 (2006).
64. Halazonetis, T. D., Gorgoulis, V. G. & Bartek, J. An oncogene-induced DNA damage model for cancer development. Science 319, 1352-1355 (2008).
65. Negrini, S., Gorgoulis, V. G. & Halazonetis, T. D. Genomic instability—an evolving hallmark of cancer. Nat Rev Mol Cell Biol 11, 220-228 (2010).
66. Bochar, D. A., et al. BRCA1 is associated with a human SWI/SNF-related complex: linking chromatin remodeling to breast cancer. Cell 102, 257-265 (2000).
67. Wang, B., et al. Abraxas and RAP80 form a BRCA1 protein complex required for the DNA damage response. Science 316, 1194-1198 (2007).
68. Sherman, M. H., Bassing, C. H. & Teitell, M. A. Regulation of cell differentiation by the DNA damage response. Trends in cell biology 21, 312-319 (2011).
69. Nikkilä, J., et al. Heterozygous mutations in PALB2 cause DNA replication and damage response defects. Nature communications 4, 2578 (2013).
70. Buchholz, T. A., et al. Evidence of haplotype insufficiency in human cells containing a germline mutation in BRCA1 or BRCA2. International journal of cancer Journal international du cancer 97, 557-561 (2002).
71. Dextraze, M.-E., Gantchev, T., Girouard, S. & Hunting, D. DNA interstrand cross-links induced by ionizing radiation: an unsung lesion. Mutation research 704, 101-107 (2010).
72. Hagen, U. Current aspects on the radiation induced base damage in DNA. Radiation and environmental biophysics 25, 261-271 (1986).
73. Nikjoo, H., O &apos; Neill, P., Wilson, W. E. & Goodhead, D. T. Computational approach for determining the spectrum of DNA damage induced by ionizing radiation. Radiation research 156, 577-583 (2001).

We claim:

1. A method of assessing the risk of developing cancer in a subject having a germline mutation in a gene known to be associated with cancer comprising:
a) providing a mitotic non-tumor cell from the subject;
b) culturing the cell to obtain a subject cell population;
c) labeling the subject cell population of step (b) with a first detectable label;
d) co-culturing the subject cell population with a control cell population labeled with a second detectable label;
e) exposing the cell populations of step (d) to a DNA damaging agent;
f) determining the ratio of cells having the first detectable label to cells having the second detectable label
wherein when the ratio is less than 1 the subject has an increased risk of developing cancer.

2. The method of claim 1, wherein the germline mutation is a BRAC1 or BRAC2 mutation.

3. The method of claim 1, wherein the non-tumor cell is a fibroblast.

4. The method of claim 3, wherein the fibroblast is a skin cell.

5. The method of claim 1, wherein the control population is derived from a subject not having the germline mutation.

6. The method of claim 1, wherein the subject cell population and the control cell population are the same type of cells.

7. The method of claim 1, wherein the DNA damaging agent is cisplatin, hydroxyurea ultraviolet radiation or 4-nitro-quinoline.

8. The method of claim 1, wherein the detectable label is a fluorescent dye.

9. The method of claim 8, wherein the ratio of the first detectable label and the second detectable label is by FACS.

10. The method of claim 1, further comprising making a clinical management recommendation for the patient.

11. The method of claim 10, wherein the clinical management recommendation is that the subject receives prophylactic therapy for said cancer.
